(19) United States
(12) Patent Application Publication
(10) Pub. No.: US 2026/0144956 A1
(43) Pub. Date: May 28, 2026

(54) MULTIPLE MODALITY HUMAN PERFORMANCE IMPROVEMENT SYSTEMS

(71) Applicant: MINDRIGHT HOLDINGS, INC., MIAMI, FL (US)

(72) Inventor: JACOB S RAUCHBACH, CHAPEL HILL, NC (US)

(73) Assignee: MINDRIGHT HOLDINGS, INC., MIAMI, FL (US)

(21) Appl. No.: 19/366,107

(22) Filed: Oct. 22, 2025

Related U.S. Application Data
(63) Continuation-in-part of application No. 18/178,080, now abandoned.
(60) Provisional application No. 63/089,200, filed on Oct. 8, 2020.

(51) Int. Cl. A61M 21/02; A61M 21/00

(52) U.S. Cl. CPC A61M 21/02; A61M 2021/0022; A61M 2021/0027; A61M 2021/0044; A61M 2205/80

(57) ABSTRACT

A system for improving human performance includes a software application with an onboarding engine, an initialization engine, an optimization engine, and a voice engine to populate a user profile, and use the profile to select and optimize a training protocol. The application is executed on a computer with processor, memory, user interface, and storage. The application exchanges information with the user and accesses a file containing the user profile. A method for improving human performance includes onboarding a new user by dynamically generating questions to create a profile; selecting an initial training protocol for the user based on the profile and verified training pathways; modifying the initial protocol based on user feedback to create an optimized training protocol, and guiding the user's training session with voice instructions that are generated by a voice engine.

### CROSS-REFERENCE TO RELATED APPLICATIONS

This application claims priority to U.S. Provisional Application No. 63/089,200, filed Oct. 8, 2020, and is a continuation-in-part of U.S. application Ser. No. 18/178,080 filed Mar. 3, 2023, which are hereby incorporated by reference herein in their entirety.

### BACKGROUND OF THE INVENTION

### Field of the Invention

Embodiments of the present invention relate, in general, to systems and methods that improve human performance in time-sensitive endeavors such as sports, performing arts, and public speaking.

### Relevant Background

In the disclosed invention, statistical performance improves when emotional, behavioral, and physical blockages are neutralized within the human energy systems. Blockages are cleared from the human energy system by way of an iterative, customized process that includes a training session curated via a mobile application, and may also include use of a wearable, or an on-line course.

The disclosed techniques are rooted in over 5,000 years of Traditional Chinese Medicine (“TCM”) as well as conventional scientific energy systems within the human body, such as the phosphagen [ATP-PC] system, glycolytic system, and oxidative phosphorylation. With respect to TCM and similar systems of medicine practiced by ancient or tribal peoples, their effectiveness must consider “psychosocial factors” not typically considered in scientific medical systems. Such medical systems relied on a “psychosocial mobilization of the patient's biochemical response system.” Further TCM and similar systems generally assume a unitary model of the human organism, wherein the mind and body are inextricably linked, rather than regarded as separate and unrelated spheres. Such a “non-reductionist theory of medical effectiveness” helps explain the observed improvements in performance, stress, and pain experience promoted by the disclosed methods and systems, which may at first glance appear outside the explanatory powers of conventional scientific medicine. See Current Anthropology, The University of Chicago Press, Vol. 20, No. 1. Pp. 59-80 (March 1979). Further, TCM and modern medical techniques may access and act upon the same underlying processes of the body, since “considerable similarities exist between ancient concepts of the life force and modern biofield concepts in their assumption that a form of life-giving energy flows throughout the body and that illness arises as a result of blockages, excesses, or irregularities in its flow.” See https://www.faim.org/measurement-of-the-human-biofield-and-other-energetic-instruments. There is a substantial body of empirical research both in and out of sports, demonstrating the efficacy of targeting TCM energy modalities to assist in human well-being.

There have been other systems to improve performance based on using TCM human energy manipulation. However, existing systems generally include only stand-alone modalities and techniques, and in practice are mostly inefficient, cumbersome, and difficult to implement. Significantly, such systems do not incorporate time as a constraint on when a performance must begin, when activities of the performance must occur, and/or how long the performance or particular elements must last. In athletics, for example, time is a crucial element for training and in-game performance. In virtually every sport, there are game clocks, shot clocks, ten-second rules, five-second rules and/or time limits. Similarly, a musical performance must occur at a set tempo, and must begin and end at a set time. These factors require a practitioner to execute the elements of the performance on the timeline demanded by the activity, or at least not solely determined by the practitioner. Therefore, time limits are a major component of performance and its improvement.

It would be desirable to have a human performance improvement system that was coherent, easy to use, that leveraged multiple different TCM modalities concurrently, and that accounted for time as a crucial aspect of performance and performance improvement. Therefore, the disclosed methods and systems use concurrent, sequential, and overlapping applications of different modalities, such as Meridian Tapping, Grounding, Shielding, and Mantras, to provide customized, multi-modality routines that powerfully and efficiently leverage time as a catalyst for progress.

Current performance training systems also suffer from being generalized across the population of users, and are not easily customized. Because training systems are static and generalized, individual users are not provided a personalized system that is tailored to their performance blockages, nor are existing systems capable of adapting over time as the user improves or does not improve their performance metrics. Further, existing protocols that are manually customized are difficult to scale, since a customized training system would require a time intensive process administered by an expert trainer. What is more, current systems are unable to adapt during a training session to provide a user with real-time adaptability. These drawbacks severely limit the efficacy of current performance improvement systems.

Therefore, whet is needed is a performance improvement system that provides one or more artificial-intelligence (AI), and/or machine learning (ML) models trained on data developed by experts in the field of performance improvement to perform intake tasks, to model a user's performance, and to generate training sessions via voice instructions. The disclosed system is able to dynamically interview users to acquire necessary information for training session customization, it autonomously matches a user's needs with a training protocol, and then generates training content that is communicated via synthetic voice instructions.

### BRIEF DESCRIPTION OF THE DRAWINGS

Features and objects of the present invention and the manner of attaining them will become more apparent, and the invention itself will be best understood, by reference to the following description of one or more embodiments taken in conjunction with the accompanying drawings and figures imbedded in the text below and attached following this description.

The Figures imbedded and attached depict embodiments of the present invention for purposes of illustration only. One skilled in the art will readily recognize from the following discussion that alternative embodiments of the structures and methods illustrated herein may be employed without departing from the principles of the invention described herein.

FIG. 1 depicts the traditional locations of meridians, colors and approximate frequencies, as used in some embodiments of the disclosed invention.

FIGS. 2A-2E depict the approximate locations of 28 foundational acupuncture and tapping points as used in some embodiments of the disclosed invention;

FIG. 3 depicts traditional chakra designations and characteristics, as used in some embodiments of the disclosed invention;

FIG. 4 depicts example pathways for a practitioner to navigate an application as used in some embodiments of the disclosed invention;

FIG. 5 is a flow chart depicting an example sequence of events in a training session used in some embodiments of the disclosed invention;

FIGS. 6A-6C depict example tapping sequences for countering stress and/or anxiety, as used in some embodiments of the disclosed invention

FIGS. 7A-7E depict example tapping sequences for countering stress and/or anxiety, as used in some embodiments of the disclosed invention;

FIGS. 8A-8C depict example tapping sequences for countering physical pain or discomfort, as used in some embodiments of the disclosed invention.

FIGS. 9A-9E depict example tapping sequences for countering physical pain or discomfort, as used in some embodiments of the disclosed invention;

FIGS. 10A-10C depict an example tapping sequence for improving performance, as used in some embodiments of the disclosed invention;

FIGS. 11A-11C depict example tapping sequences for countering frustration or anger, as used in some embodiments of the disclosed invention;

FIGS. 12A-12E depict example tapping sequences for countering frustration or anger, as used in some embodiments of the disclosed invention;

FIGS. 13A-13C depict example tapping sequences for improving confidence, as used in some embodiments of the disclosed invention;

FIGS. 14A-14E depict example tapping sequences for improving confidence, as used in some embodiments of the disclosed invention;

FIGS. 15A-15E depict example tapping sequences for improving confidence, as used in some embodiments of the disclosed invention;

FIGS. 16A-16D depict an example tapping sequence for high vibe, as used in some embodiments of the disclosed invention;

FIGS. 17A-17E depict an example tapping sequence for enhancing a feeling of calm, as used in some embodiments of the disclosed invention;

FIGS. 18A-18E depict an example tapping sequence for countering pain or discomfort, as used in some embodiments of the disclosed invention;

FIGS. 19A-19B depict an example tapping sequence for use during performance of activities, as used in some embodiments of the disclosed invention;

FIG. 20 is an example diagram illustrating approximate frequencies associated with various emotions or mental states, as used in some embodiments of the disclosed invention;

FIG. 21 depicts example wearable devices to be used in some embodiments of the disclosed invention.

FIG. 22 is a block diagram showing an exemplary system architecture as used in embodiments of the disclosed invention.

FIG. 23 is a flow chart showing an exemplary onboarding procedure as used in embodiments of the disclosed invention.

FIG. 24 is a flow chart showing an exemplary AI/ML feedback loop as used in embodiments of the disclosed invention.

FIG. 25 is a block diagram showing an exemplary computer system as used in embodiments of the disclosed invention.

### DEFINITIONS

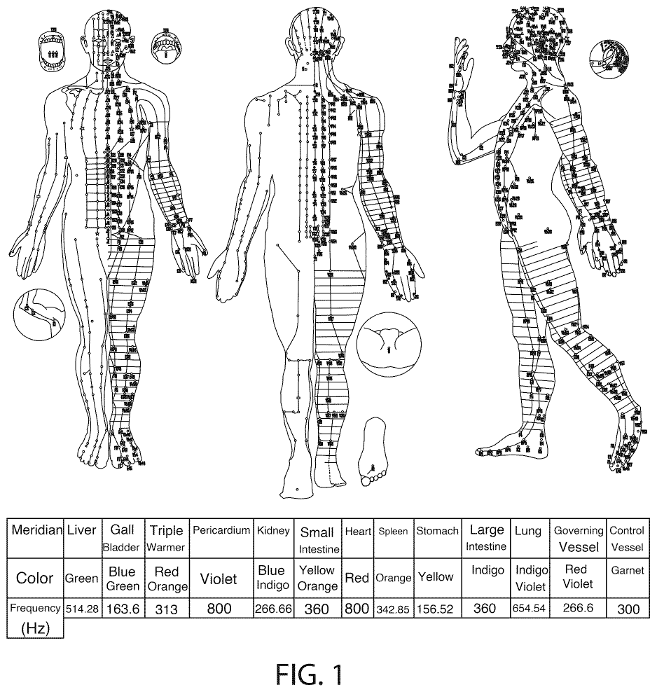

As used herein, meridian means one of the life energy, or qi, pathways located within the human body as known in Traditional Chinese Medicine. While there are no anatomical structures corresponding to meridians, their function in TCM facilitates the movement of life energy throughout the body. With reference to FIG. 1, the meridians include 12 principal meridians, and 8 extraordinary meridians.

As used herein, standard, or principal meridian means one of the following 12 meridians, each of which is associated with an organ: bladder, gall bladder, stomach, kidney, spleen, liver, lung, large intestine, pericardium, heart, small intestine, and triple warmer. Principal meridians are bilaterally symmetrical and are associated with acupuncture points on the body. The triple warmer is a meridian identified in TCM, but lacks a scientific anatomical counterpart.

As used herein, extraordinary meridian means one of 8 meridians, each of which is associated with a TCM energy storage reservoir within the human body. Relevant to the disclosed invention are the Yin Wei Mai or Yin Linking Vessel, and the Yang Wei Mai or Yang Linking Vessel.

As used herein, a midline meridian is an extraordinary meridian that intersects one or more of the principal meridians and is associated with acupuncture points on the body. The midline meridians include the Governing Vessel and Conception/Central Vessel.

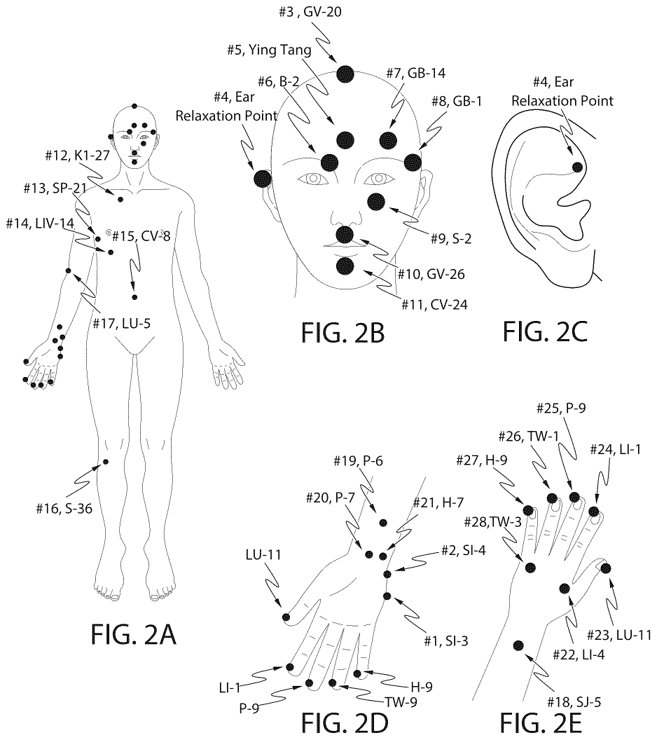

As used herein, an acupuncture point is one of several hundred locations on the human body used in TCM for the treatment of pain and other conditions. Each acupuncture point is located along one or more principal or midline meridians. With reference to FIG. 2, relevant to the disclosed invention are 28 of these acupuncture points, each of which has a designated location on the human body, and corresponds to a function of the disclosed invention.

As used herein, human energy system means the system of meridians, organs, chakras, acupuncture points, and vessels that facilitate the flow of life energy throughout the body.

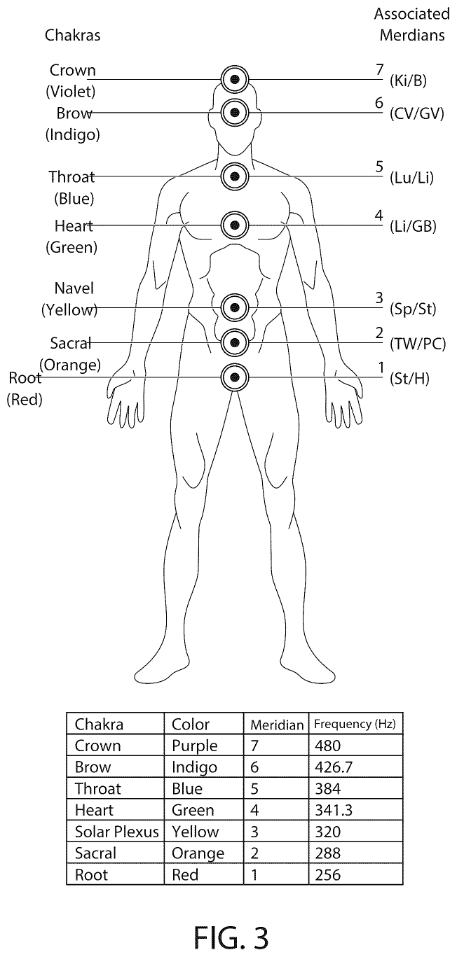

As used herein, a chakra is a focal point for human energy that originated in traditional Hindu meditation practices. Chakras may be associated with locations on the human body, meridians, colors, and frequencies. See FIG. 3.

As used herein, body mapping means a process for characterizing pain or discomfort in a practitioner's body that includes having the practitioner: 1) identify where in their body the pain exists, 2) indicate how intense the pain is, and 3) assign a shape and/or color to the pain.

As used herein, tapping means manipulation of acupuncture points that are located on meridians on the human body for promoting emotional, behavioral, physical, or other performance effects in a practitioner. Such manipulation may include tapping, rubbing, or touching the skin surface at locations corresponding to the acupuncture points. Tapping may be used as a stand-alone procedure, or may be combined with other disclosed procedures, such as grounding, shielding, or mantras.

As used herein, In-Performance Tapping is an abbreviated tapping procedure tailored for in-game or in-performance use and is intended to be performed within the time frame available to the practitioner. Foul Line Tapping is a specialized version of the In-Performance Tapping procedure for use during basketball foul shooting or other performance situations, both in and out of sport.

As used herein, a primer statement is a statement of self-acceptance used by a practitioner in conjunction with a tapping procedure.

As used herein, grounding is a procedure to allow the practitioner to energetically and emotionally stabilize themselves to improve focus and increase performance. There are at least two versions of grounding, including a Long Grounding, which takes from 1 to 10 minutes, for use during periods wherein the practitioner has more relaxed time constraints; and Performance Grounding, which takes 3 to 59 seconds, for use during time-constrained periods. Grounding may be used as a stand-alone procedure, or may be combined with other disclosed procedures.

As used herein, a mantra or affirmation is a procedure for using positive statements that allows the practitioner to reinforce positive emotions, mental states, behavior, physical sensations, and/or performance. Each mantra targets specific emotions and behaviors, which directly correspond to the meridian system. For example, anger is traditionally associated with the liver meridian. Therefore, a mantra for counteracting a feeling of anger in the practitioner would involve the liver meridian and positive statements that are inversely related to anger. Mantras may be used as a stand-alone procedure or may be combined with other disclosed procedures.

As used herein, a positive emotion, mental state, behavior, or physical sensation is one that tends to improve performance by the practitioner in the context of an activity. For example, the mental state of nervousness felt prior to a basketball game may be considered negative, whereas the state of excitement may be considered positive in that context.

As used herein, shielding is a procedure that allows the practitioner to create an emotional and behavioral barrier that serves to counteract outside stimuli that can potentially impede the practitioner's focus, and thus their performance. There are at least two versions of shielding including a Long Shielding, which takes from 1 to 3 minutes, for use during periods wherein the practitioner has less time constraints; and Performance Shielding, which takes 3 to 59 seconds, for use during time-constrained periods. Shielding may be used as a stand-alone procedure or may be combined with other disclosed procedures.

As used herein, symbolling is the use of visual representations that have either inherent or acquired significance for the practitioner, wherein the symbol signifies and promotes emotional, behavioral, physical, or other performance effects in the practitioner. For example, the practitioner may be shown a Yin-Yang symbol to signify emotional balance, or a representation of a spirit animal or mascot to promote confidence or competitiveness, etc.

As used herein, auditory frequency means the application of sound waves to the ears or other parts of the human body to achieve emotional, behavioral, physical, or other performance effects in a practitioner. Auditory frequency may be music, such as classical music, or may be a specific frequency or range of frequencies selected to neutralize or enhance particular emotions, mental states, or energies.

As used herein, frequency means either a frequency or range of frequencies associated with an energy center or chakra, a meridian, an organ, or a behavioral or emotional state. Frequency may also mean a frequency or range of frequencies that achieves an effect on a chakra, meridian, organ, or a behavioral or emotional state, e.g., enhancement or neutralization. Each meridian or chakra has an optimal frequency at which it operates. When that frequency is blocked, there is a range of effective frequencies for each meridian or chakra that can be applied to the body to promote removal of the blockage and bring the element back into balance. What follows is one example of the frequencies associated with specific organs and emotions/mental states within the meridian system:
Lung: 2287 Hz
Large intestine: 4230 Hz
Stomach: 126.9 Hz
Spleen: 264.9 Hz
Heart: 289 Hz
Small intestine: 316 Hz
Bladder: 343.8 Hz
Kidney: 383.7 Hz
Pericardium: 477 Hz
Triple Warmer: 496 Hz
Gall Bladder: 506.8 Hz
Liver: 1032 Hz
   The effective range for promoting energy balance for a particular meridian and specific situation is yet to be determined, and may vary based on the requirements of a participant or situation. An additional example of frequency ranges associated with meridians is depicted in FIG. 1, while an exemplary list of associated frequencies for chakras is depicted in FIG. 3.

As used herein, color therapy means the application of a color or a sequence of colors to a practitioner's visual field to promote emotional, behavioral, physical, or other performance effects in the practitioner. In TCM, a color may be associated with an energy center or chakra, a meridian, an organ, or a behavioral or emotional state.

As used herein, color light therapy, or chromotherapy, means use of colors of light, each of which has a specific frequency to affect the human body and its functioning. Light is able to penetrate from the epidermis through the vascular system to the cellular level of the human body.

As used herein, psycho-somatic means the negative or positive impact that emotions or mental states have on the physical condition and overall health of the human body. Other terms used traditionally for this concept are prana, chi, aura, energy, and orgone.

As used herein, vibration means frequencies, forces or motions applied to the human body, specifically the skin and in some cases underlying tissue, to promote emotional, behavioral, physical, or other performance effects in the practitioner.

As used herein, biofeedback means tracking physiological data through wearable or other sensors, comprising heart rate, heart rate variability, respiratory variability, pulse oximetry, galvanic skin response, temperature, facial coding, electrocardiography (EKG or ECG), electroencephalography (EEG), electromyography (EMG), electrooculography (EOG), or other relevant metric.

Artificial intelligence (AI) means the use of computers to emulate human cognitive functions. AI therefore refers to the use of machines to accomplish tasks via algorithms in a manner similar to human intelligence. Machine learning (ML) means a subset of AI wherein machines execute algorithms allowing the machines to receive a set of data, learn from the data, and change algorithms based on the information learned.

AI, and ML each refer to multiple techniques rather than a single method of computing.

### DETAILED DESCRIPTION OF THE INVENTION

The detailed description of the present invention will be primarily, but not entirely, limited to systems, methods, and sub-methods for improving human performance in contexts in which time is a critical factor. Using a suite of complementary modalities, this invention leverages both TCM and conventional scientific energy systems to synthesize previously complicated and fragmented methods into one system of performance improvement. Governed by time limits, the disclosed system allows practitioners to optimize performance by neutralizing mental and energetic blockages/disruptions in the human body's energy systems.

The systems and methods of human performance improvement disclosed herein were developed through extensive experience unlocking human performance. In particular, the disclosed methods and systems have been shown to promote statistically significant improvement in performance, reduction in reported stress and anxiety, and reduction in the perception of physical pain for athletes and teams in professional and college basketball. The benefits, however, are not limited to basketball, but extend to various other sports, and reach across multiple disciplines, including the performing arts, public speaking, or any other activity with time-critical aspects.

The techniques disclosed herein are implemented through a system comprising software running in a mobile application hosted on a smartphone, laptop, tablet, desktop, or other computing device, and in some embodiments may be complemented by use of a wearable device. On-line data analysis and storage, as well as online live or recorded coaching programs may also be included. The system reduces stress, anxiety, physical pain, discomfort, frustration, anger, low confidence, insecurity, lack of focus, lack of energy, and improves performance by targeting and removing emotional, behavioral, or physical blocks experienced by practitioners of the techniques.

Embodiments of the present invention are hereafter described in detail with reference to the accompanying Figures. Although the invention has been described and illustrated with a certain degree of particularity, it is understood that the present disclosure has been made only by way of example and that numerous changes in the combination and arrangement of parts can be resorted to by those skilled in the art without departing from the spirit and scope of the invention.

The following description with reference to the accompanying drawings is provided to assist in a comprehensive understanding of exemplary embodiments of the present invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. Also, descriptions of well-known functions and constructions are omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

Like numbers refer to like elements throughout. In the figures, the sizes of certain lines, layers, components, elements or features may be exaggerated for clarity.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. Thus, for example, reference to “a component surface” includes reference to one or more of such surfaces.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a nonexclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be also understood that when an element is referred to as being “on,” “attached” to, “connected” to, “coupled” with, “contacting”, “mounted,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of a device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of “over” and “under”. The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

### Application Pathway and Training Structure

A practitioner interacts with the system through an inquiry process, either manually with an in-person instructor, via an application hosted on a computing device, or a application-based training session guided by an instructor. The inquiry process determines the practitioner's performance-related goals, blockages, desired outcomes, and may seek input relevant to the practitioner's human energy status. Successive levels of inquiry and practitioner responses are used to generate a customized training session recommending the execution of one or more modalities, comprising tapping of acupunctural meridians, grounding routines, mantra recitation, and shielding routines. These modalities may also be combined with one or more therapies, comprising frequency, vibration, music, color, and light therapies. Therapies may be administered by a companion wearable device configured to generate and infuse therapies into the body. The resulting combination acts to neutralize the blockages within human body's energy systems, creating a context for improved performance.

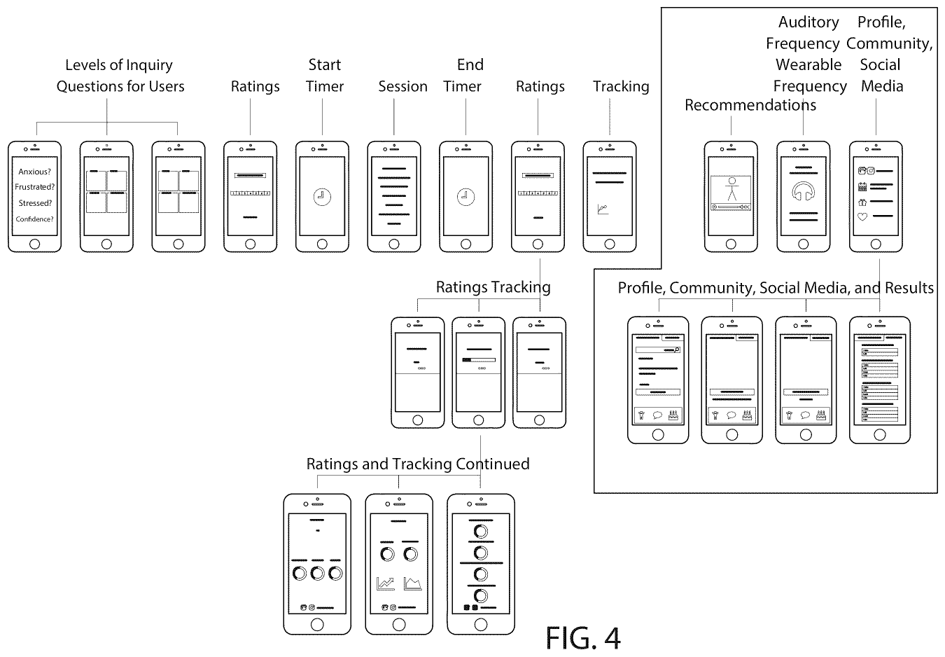

With reference to FIG. 4, embodiments of the disclosed systems and methods include an application configured to run on a computing device, such as a smartphone, tablet, laptop, desktop, or other device. The computer configured to run the application includes aspects commonly known in the art, including a central processor, working memory for the processor, a visual display, an input means, such as a mouse, keyboard, and/or touchscreen, and data storage means. The application includes pages that are accessible on a display of the computing device, and which provide information to and solicit input from the application user.

The application creates a profile for each practitioner, and builds the profile by soliciting input on several relevant fact categories, which may include the practitioner's identity, location, age, field of endeavor (i.e., public speaking, athletics, performing arts, etc.), sport, activity, performance goals, training goals, physical fitness, emotional state, suspected emotional blockages, suspected physical blockages, and so on. Practitioners may also use the application to message, communicate, and/or interact with other practitioners or users of the application. Aspects of this invention may also include social media, question and comment boards, individual and collective scoreboards, ratings systems, and/or reward systems.

Once a practitioner has completed their profile, the application will begin an inquiry to solicit responses to allow the application to direct the practitioner to a training session. The application may select and direct the inquiry by use of an algorithm and/or based on input from the practitioner, or another application user, such as a coach or physical therapist, that is directing the practitioner's training. The inquiry may include one or more levels. A first level of inquiry may prompt the user to make a number of simplistic choices relevant to an emotional condition, mental state, a physical condition, a list of questions, a survey, a questionnaire, and/or physical testing. Subsequent levels of inquiry, if used, will seek input based on the user's responses to the previous level of inquiry. Subsequent levels of inquiry may use the same, a subset of, or additional categories of input, in order to facilitate the selection of a suitable training session for the user. Based upon the results of the one or more levels of inquiry, a training session comprising one or more sequences or modalities is selected. The training session may be selected by the application, or a user, to address areas including emotional, behavioral, performance, and/or physical blocks. The training session may be generated by the application based on practitioner or other user input, or may be selected from a library, the library being a pre-existing set of customized or customizable training modalities or sequences that are accessible by the application.

Once a training session is selected, the application prompts the practitioner to perform one or more sequences of modalities, e.g., tapping, shielding, grounding, and/or mantras, through instructions displayed on the device. In concert with the performed modality sequence(s), the application may also transmit instructions to one or more wearable devices which transmits an input to the practitioner. Such inputs may include one or more of an audible frequency, music, vibration, color, light, or other suitable input. As the practitioner moves through the guided modality sequence, the application will provide on-screen instructions and suggestions, and in some embodiments will administer frequency, vibratory, and/or visible light inputs through the wearable(s). By curating the practitioner's training session and administering complementary inputs from the wearable device(s), the training session efficiently identifies and promotes the neutralization of energetic, emotional, or behavioral issues that may be impeding the practitioner's performance.

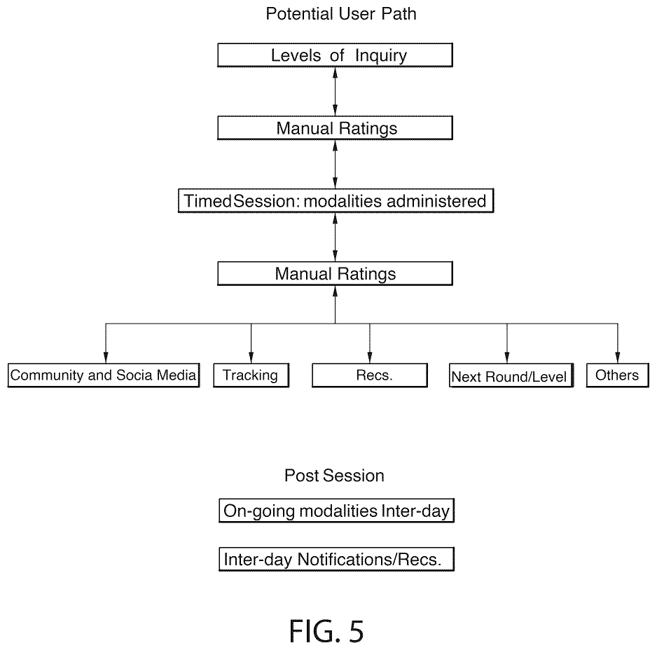

Once the practitioner completes or otherwise ends the recommended training session, the application guides the practitioner through a post-training phase. With reference to FIG. 5, after the training session, the application may present the practitioner with options based on practitioner, other user, or application programming. For example, the application may end training for the day or other period of time. Alternatively, the application may initiate a subsequent training session. The follow-on training session may be selected based at least in part on the practitioner's execution of, or reaction to, the prior training session, and may include one or more follow-up levels of inquiry. In some cases, the application will move the practitioner to a suite of post-training activities.

In some embodiments, during the post-training phase, the application may solicit feedback from the practitioner or other user regarding the training session. Such feedback may include a rating or satisfaction score for the training session, such as how well the training session improved the practitioner's blockage, ease of use, the practitioner's post-training mental or physical state, the practitioner's performance in their activity, or other criteria. The application may also track whether the practitioner performed the full recommended training sequence or only a portion. In some embodiments, the application stores at least a portion of the information gathered during and after the training session in the practitioner's profile. Such information may include practitioner or other user responses and inputs to the application inquiries, feedback, training session completion status, etc. The practitioner's profile as updated may then be used as inputs for future training session recommendations. The application may also provide recommendations to the practitioner in the form of status updates, notifications, reminders, text messages, emails, etc. These recommendations may include suggested follow-on sequences of modalities, and may also provide prescriptions and ongoing support to the practitioner as they work through the recommended sequences. Post-session training may also include inputs by the wearable device(s) either in coordination with other modalities or as stand-alone inputs for the practitioner. On-line coaching programs also may be provided.

### Training Modalities and Inputs

A primary component of training sessions of the disclosed invention is the practitioner's performance or experience of one or more modalities. These include, but are not limited to, the following: tapping of acupunctural meridians, grounding, mantras, shielding, audible frequencies, music, color, and light. In-concert with the modalities, in some embodiments another component of the training session is frequency and vibration inputs administered by the application through one or more wearable devices worn by the practitioner. The device(s) will input multiple and varying frequencies, including but not limited to: audible frequencies, music, vibrations, radio waves, light, color, and sound to neutralize the emotional or behavioral blockages within the practitioner's energy systems. Modalities and sequences comprising the training sessions are optimized to address specific performance blockages, with the intention to amplify mental or physical well-being, and the performance improvement impact of the training session. While certain modalities and sequences are optimized for specific blockages, they may be used to clear other blockages depending on the needs of the practitioner. For example, a sequence designed for clearing a blockage of stress or anxiety may be used in some cases to clear a blockage of anger or frustration or lack of focus. Generally, however, sequences designed to clear physical blockages are not used to clear emotional blockages, and vice versa. Some sequences and modalities are executed under time constraints to correspond to time-critical requirements of a performance or activity.

Tapping is a modality of the disclosed invention that promotes neutralization of energetic disruption through manipulating acupuncture points on the human body. With further reference to FIG. 2, the acupuncture points used in tapping are shown with locations indicated on the body. Each point is assigned a number for identification within a tapping sequence as assigned to the practitioner. Further, FIG. 2 indicates meridians or organs associated with each point. Many of the identified acupuncture points have a bilaterally symmetric counterpart that are also leveraged by the systems herein. For example, Point #16 shown on the right knee has a counterpart located in the corresponding location on the left leg. A unique aspect of tapping modalities described herein is the combination of points manipulated, as well as the sequential order of manipulation.

During the inquiry portion of a training program, the practitioner will identify categories of distress experienced or selected to address and record their level of distress in standardized units. Distress categories may include emotional, behavioral, performance, or chronic injury or pain issues. Tapping sequences may also target stress, anxiety, frustration, anger, or confidence as areas in which to seek improvement. Once the distress categories or targets for improvement are identified and baseline level of distress are established, the application, either working from an algorithm, or based on inputs from the practitioner or other user, will select one or more rounds of specific tapping sequences for the practitioner to perform. Tapping sequences will generally be a subset of 28 foundational tapping points identified in FIG. 2. An exemplary listing of these 28 foundational points follows:

### Foundational Tapping Points

1) Small Intestine: SI-3 (Side of Wrist)
2) Small Intestine: SI-4 (Side of Wrist)
3) Governing Vessel: GV-20 (Top of Head)
4) Ear Relaxation Point (No Meridian): (Top of Ear)
5) Extra Point (No Meridian): Ying Tang Extra Point #1 (Between Eyebrows)
6) Bladder: B-2 (Eyebrow)
7) Gall Bladder: GB-14 (Above Middle of Eyebrow)
8) Gall Bladder: GB-1 (Side of Eye)
9) Stomach: S-2 (Under Eye)
10)Governing Vessel: GV-26 (Under Nose)
11)Central Vessel: CV-24 (Chin)
12)Kidney: KI-27 (Collarbone)
13)Spleen: SP-21 (Side of Rib)
14)Liver: LIV-14 (Under Chest)
15)Central Vessel: CV-8 (Navel)
16)Stomach: S-36 (Knee)
17)Lung: LU-5 (Inner Elbow)
18)Yang Wei Mai (Yang Linking): SJ-5 (Top Mid Arm)
19)Yin Wei Mai (Yin Linking): P-6 (Bottom Mid Arm)
20)Pericardium: P-7 (Middle Bottom Wrist)
21)Heart: H-7 (Outside Bottom Wrist)
22)Large Intestine: LI-4 (Thumb—Index Web)
23)Lung: LU-11 (Thumb)
24)Large Intestine: LI-1 (Index Finger)
25)Pericardium: P-9 (Middle Finger)
26)Triple Warmer: TW-1 (Ring Finger)
27)Heart: H-9 (Pinky)
28)Triple Warmer: TW-3 (gamut)

Tapping sequences based on the 28 foundational points may use only a subset of the points, and/or may manipulate the points in a different prescribed sequence. Where multiple rounds of tapping sequences are used, the training session will address a range of emotions, states, and/or behaviors, which are progressively clarified in each round to reach improved performance. The number of rounds used may be increased or decreased as the training session progresses, and as determined by the needs and progress of the practitioner.

There are two main categories of sequences: Session Protocols, and Special Use or Abbreviated Protocols. Session Protocols include sequences to address Confidence, Anger/Frustration, Physical Pain, and Stress and/or Anxiety. Sessions comprise three rounds of tapping followed by grounding techniques and then shielding techniques at the end of the third round of tapping. The First and Third Rounds of tapping point sequences are the same within each session. Special Use Protocols include only one round of tapping points, which may or may not be cycled through multiple times during the session, until a set of grounding techniques followed by shielding techniques are included at the end of the last cycle of tapping.

In another embodiment, the first tapping round may use a primer statement, i.e., a statement of self-acceptance uttered by the practitioner. A primer statement is selected based on an emotional, behavioral, or psycho-somatic element or blockage specifically targeted by the tapping sequence for neutralization. For example, a primer statement targeting stress or anxiety may be “Even though I feel stressed, I can now relax,” or “Even though I feel anxious, I deeply and completely love myself.” With further reference to FIG. 2, the practitioner will use the primer statement in conjunction with manipulating Point #1, the first point tapped in the sequence. The practitioner may elect to keep the topic broad, or may also insert a specific blockage event or memory from their life into the primer statement. For example, “Even though I feel anxiety from my car accident, I can now relax.” The practitioner may then move through the tapping sequence, either without further use of the primer statement, the repetition of an abbreviated primer, e.g., “This anxiety,” or the repetition, either silently or aloud, of the full primer statement. Use of the primer statement will be coordinated with tapping each point in the prescribed sequence.

Primer statements may be used to initiate rounds of tapping sequences, and are typically preset by the administrator of the sequences, by the application, etc. Not every round of tapping will initiate with a primer statement, and in particular the third round of most sessions will not start with a primer. Special Use Protocols will typically not include primer statements. In some embodiments, practitioners can designate and use their own primer statements along with their own tapping point sequences to create their own customized sessions.

In another example, a basketball player targeting stress as a blockage may use the primer statement “Even though I feel stressed because of my free throw shooting ability, I can now relax.” An abbreviated primer may then be “This stress that I feel.” As the practitioner moves to subsequent rounds of tapping sequences, the primer statement or abbreviated primer may change as new blockages become known. Primer statements may change from one tapping point to the next, or may change from round to round or training session to training session.

In contrast to the disclosed method of tailored tapping sequences, which are used in coordination with other modalities and strictly circumscribed by time constraints, existing tapping techniques typically use a universal tapping sequence that is not time constrained, and not used with other modalities. An example existing universal tapping sequence is the following

### Universal Sequence #1:

1) Small Intestine: SI-3 (Side of Hand)
2) Governing Vessel: GV-20 (Top of Head)
3) Bladder: B-2 (Eyebrows)
4) Gall Bladder: GB-1 (Side of Eye)
5) Stomach: S-2 (Under Eye)
6) Governing Vessel: GV-26 (Under Nose)
7) Central Vessel: CV-24 (Chin)
8) Kidney: KI-27 (Collarbone)
9) Spleen: SP-21 (Side of Rib)
10)Liver: LIV-14 (Under Chest)
11)Lung: LU-11 (Side of Thumb)
12)Large Intestine: LI-1 (Index Finger)
13)Pericardium: P-9 (Middle Finger)
14)Heart: H-9 (Pinky)
15)Triple Warmer: TW-3 (gamut)

Compared to tapping sequences employing the 28 foundational points, existing tapping systems activate fewer meridians. The universal sequence above includes 12 ordinary meridians and 2 centerline meridians. However, sequences using the 28 foundational points activate the 12 ordinary meridians, the 2 centerline meridians, and 2 to 6 extraordinary meridians. The extraordinary meridians activated by the disclosed tapping methods include both the Yin Wei meridian and the Yang Wei meridian, which have not been used in existing tapping systems. Further, the disclosed tapping sequences activate multiple points on certain meridians, where existing tapping sequences only activate a single point per meridian. Existing sequences are also constructed from as many as 360 and as few as 13 different acupuncture points, where the disclosed tapping sequences consistently use all or some of the 28 points.

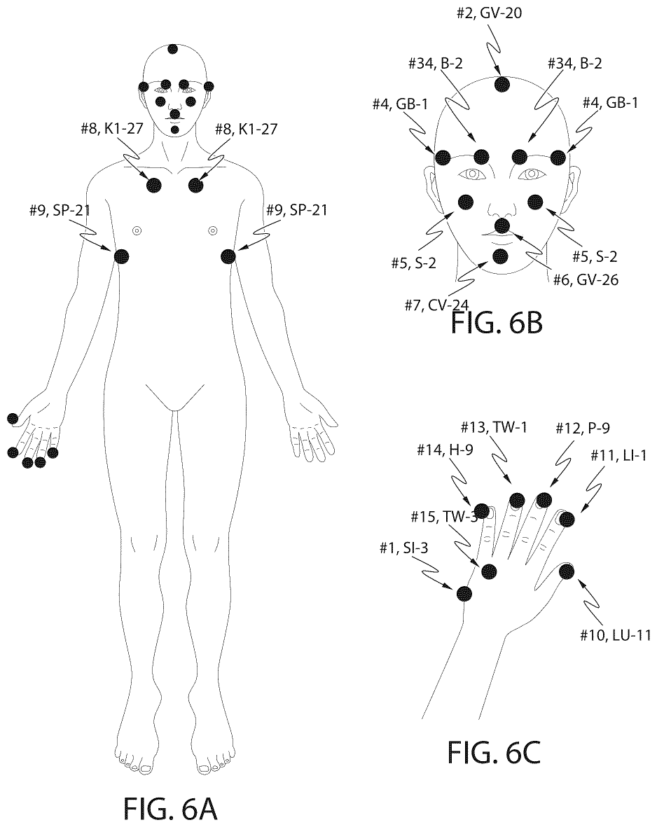

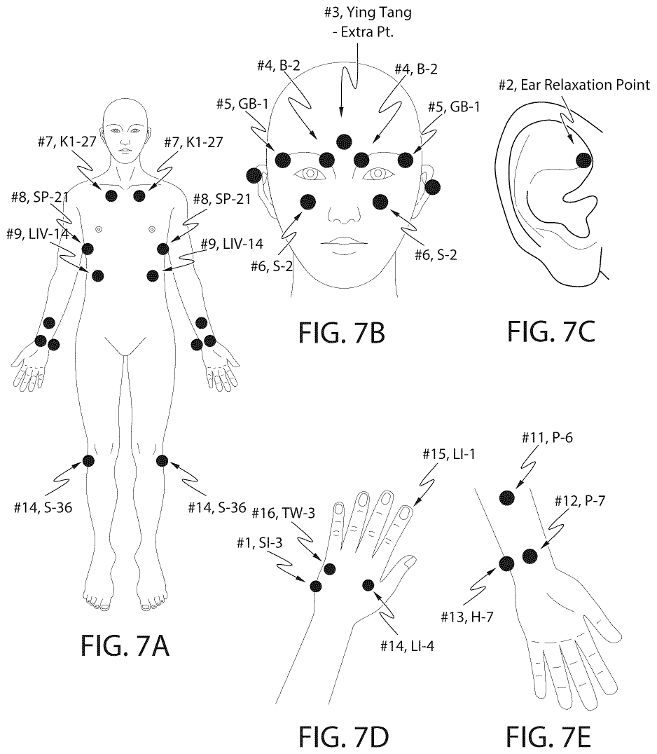

An example session targeting the blockage of stress and/or anxiety will now be described using the stress/anxiety algorithm. In this example, the application recommends a training session comprising three rounds of tapping sequences. With reference to FIG. 6, Round One includes a 15-point tapping sequence. With reference to FIG. 7, Round Two includes a unique tapping sequence using 15 different tapping points derived from the 28 foundational points, and activates meridians associated with the following: gall bladder, triple warmer, kidney, bladder, small intestine, spleen, stomach, large intestine, liver, pericardium, heart, yin linking, and uses the extra ear relaxation point, and Ying Tang point between the eyebrows. Round Three uses the same 15-point tapping sequence used in the first round, concluding with a grounding sequence followed by a shielding sequence at the end of the round. Some versions may be executed under strict time limits, such as those imposed by social media applications like Snap Chat. Mantras and affirmations maybe used in any of the three rounds. The three rounds of tapping sequences, with grounding and shielding techniques are summarized below.

Stress and/or Anxiety Training Session

### Round #1

1) Small Intestine: SI-3 (Side of Hand)
2) Governing Vessel: GV-20 (Top of Head)
3) Bladder: B-2 (Eyebrow)
4) Gall Bladder: GB-1 (Side of Eye)
5) Stomach: S-2 (Under Eye)
6) Governing Vessel: GV-26 (Under Nose)
7) Central Vessel: CV-24 (Chin)
8) Kidney: KI-27 (Collarbone)
9) Spleen: SP-21 (Side of Rib)
10)Lung: LU-11 (Thumb)
11)Large Intestine: LI-1 (Index Finger)
12)Pericardium: P-9 (Middle Finger)
13)Triple Warmer: TW-1 (Ring Finger)
14)Heart: H-9 (Pinky Finger)
15)Triple Warmer: TW-3 (Gamut Point)

### Round #2

1) Small Intestine: SI-3 (Side of Hand)
2) Ear: Relaxation Point (Top of Ear)
3) Extra Point: Ying Tang Point (Between Eyebrows)
4) Bladder: B-2 (Eyebrow)
5) Gall Bladder: GB-1 (Side of Eyebrow)
6) Stomach: S-2 (Under Eye)
7) Kidney: KI-27 (Collarbone)
8) Spleen: SP-21 (Side of Rib)
9) Liver: LIV-14 (Under Chest)
10)Stomach: S-36 (Knee)
11)Yin Wei Mai (Yin Linking): P-6 (Bottom Mid Arm)
12)Pericardium: P-7 (Middle of Wrist)
13)Heart: H-7 (Outside Bottom Wrist)
14)Large Intestine: LI-4 (Thumb—Index Web)
15)Triple Warmer: TW-3 (Gamut)

### Round #3

1) Small Intestine: SI-3 (Side of Hand)
2) Governing Vessel: GV-20 (Top of Head)
3) Bladder: B-2 (Eyebrow)
4) Gall Bladder: GB-1 (Side of Eye)
5) Stomach: S-2 (Under Eye)
6) Governing Vessel: GV-26 (Under Nose)
7) Central Vessel: CV-24 (Chin)
8) Kidney: KI-27 (Collarbone)
9) Spleen: SP-21 (Side of Rib)
10)Lung: LU-11 (Thumb)
11)Large Intestine: LI-1 (Index Finger)
12)Pericardium: P-9 (Middle Finger)
13)Triple Warmer: TW-1 (Ring Finger)
14)Heart: H-9 (Pinky Finger)
15)Triple Warmer: TW-3 (Gamut Point)
16)Grounding Technique

### 17)Shielding Technique

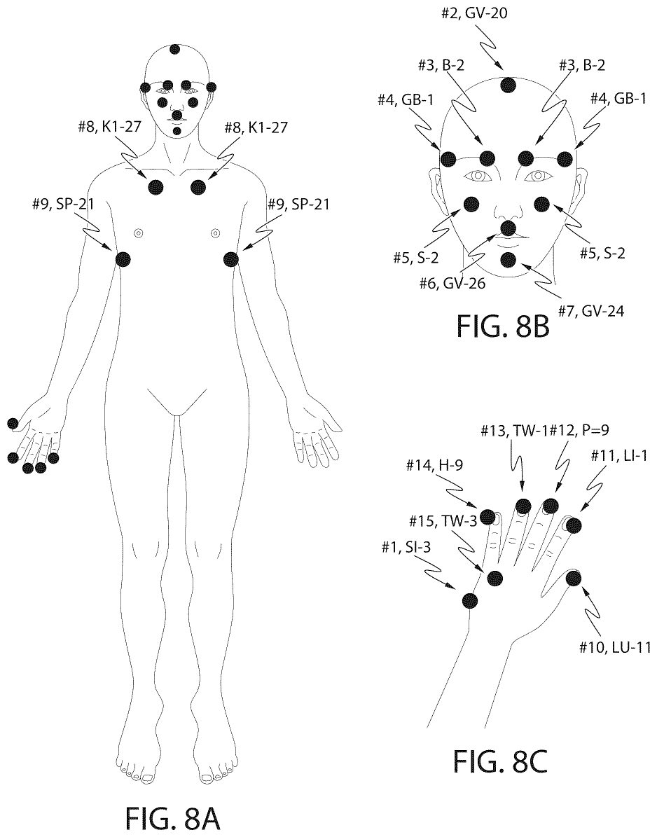

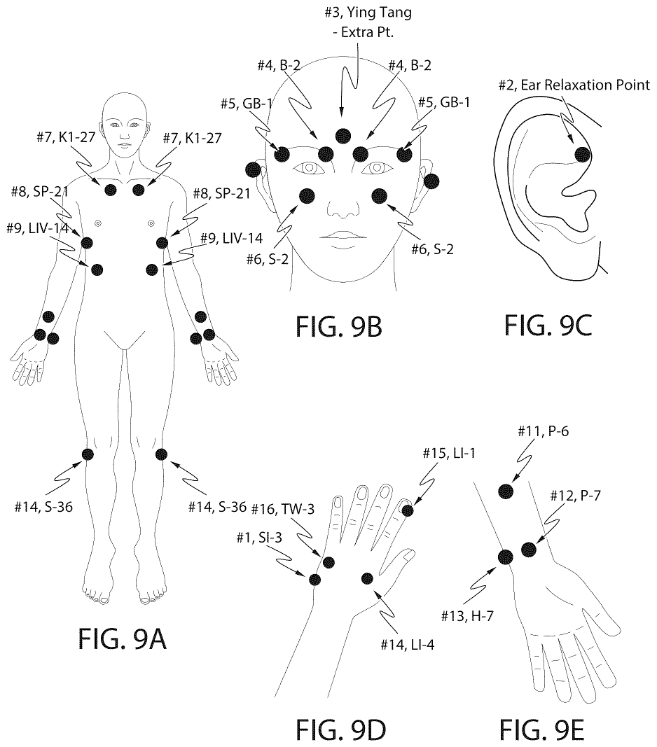

An example session of the disclosed invention targeting the blockage of physical pain, injury recovery, or physical discomfort using the Physical discomfort/Pain specific algorithm will now be described. In this example, the application recommends a training session comprising three rounds of tapping sequences. With reference to FIG. 8, Round One includes a 15-point tapping sequence. With reference to FIG. 9, Round Two includes a unique tapping sequence using 16 different tapping points, based upon the 28 foundational points, and activates meridians associated with the following: gall bladder, triple warmer, kidney, bladder, small intestine, spleen, stomach, large intestine, liver, pericardium, heart, Yin linking, and uses the extra ear relaxation point, and Ying Tang point between the eyebrows. Round Three uses the same 15 point tapping sequence used in the first round, incorporating a grounding sequence followed by a shielding sequence at the end of the round. Some versions may be executed under strict time limits, such as those imposed by social media applications. Mantras and affirmations maybe used in any of the three rounds. The three rounds of tapping sequences, with grounding and shielding techniques are summarized below.

Physical Pain and/or Discomfort Training Session

### Round #1

1) Small Intestine: SI-3 (Side of Hand)
2) Governing Vessel: GV-20 (Top of Head)
3) Bladder: B-2 (Eyebrows)
4) Gall Bladder: GB-1 (Side of Eye)
5) Stomach: S-2 (Under Eye)
6) Governing Vessel: GV-26 (Under Nose)
7) Central Vessel: CV-24 (Chin)
8) Kidney: KI-27 (Collarbone)
9) Spleen: SP-21 (Side of Rib)
10)Lung: LU-11 (Thumb Finger)
11)Large Intestine: L-1 (Index Finger)
12)Pericardium: P-9 (Middle Finger)
13)Triple Warmer: TW-1 (Ring Finger)
14)Heart: H-9 (Pinky)
15)Triple Warmer: TW-3 (Gamut)

### Round #2

1) Small Intestine: SI-3 (Side of Hand)
2) Ear Relaxation Point
3) Ying Tang: Extra Point
4) Bladder: B-2 (Eyebrows)
5) Gall Bladder: GB-1 (Side of Eyes)
6) Stomach: S-2 (Under eyes)
7) Kidney: KI-27 (Collarbone points)
8) Spleen: SP-21 (Side of Ribs)
9) Liver: L-14 (Under Chest)
10)Stomach: S-36 (Side of knees)
11)Pericardium: P-6 (Inside lower forearm)
12)Pericardium: P-7 (Wrist)
13)Heart: H-7 (Inside bottom wrist)
14)Large Intestine: LI-4 (Webbing of Thumb)
15)Large Intestine: LI-1 (Index Finger)
16)Triple Warmer: TW-3 (Gamut)

### Round #3

1) Small Intestine: SI-3 (Side of Hand)
2) Governing Vessel: GV-20 (Top of Head)
3) Bladder: B-2 (Eyebrows)
4) Gall Bladder: GB-1 (Side of Eye)
5) Stomach: S-2 (Under Eye)
6) Governing Vessel: GV-26 (Under Nose)
7) Central Vessel: CV-24 (Chin)
8) Kidney: KI-27 (Collarbone)
9) Spleen: SP-21 (Side of Rib)
10)Lung: LU-11 (Thumb Finger)
11)Large Intestine: L-1 (Index Finger)
12)Pericardium: P-9 (Middle Finger)
13)Triple Warmer: TW-1 (Ring Finger)
14)Heart: H-9 (Pinky)
15)Triple Warmer: TW-3 (Gamut)
16)Grounding Technique
17)Shielding Technique

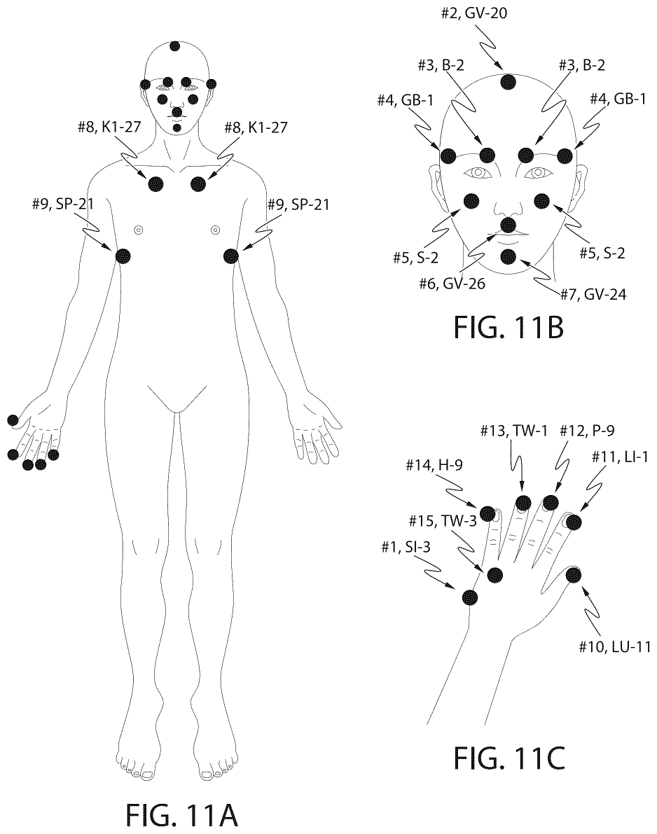

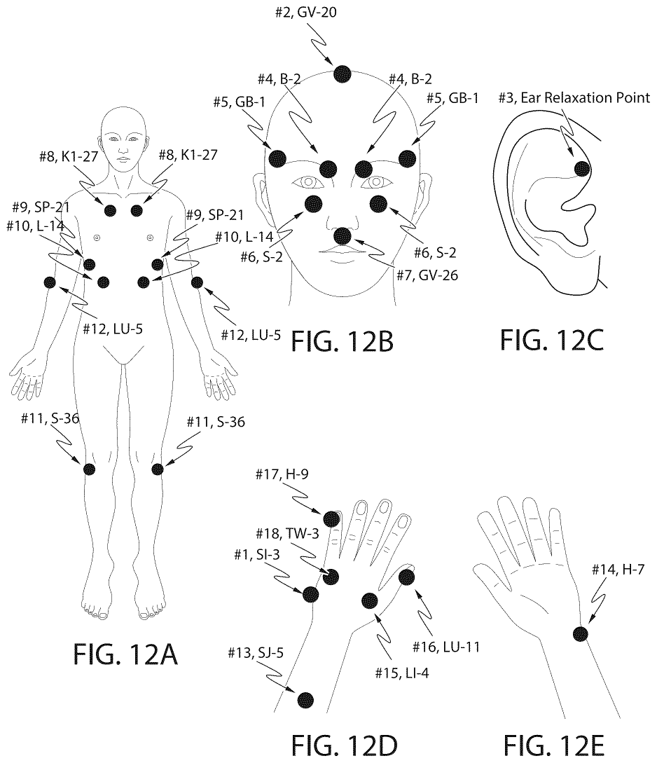

An example session of the disclosed invention targeting the blockage of frustration will now be described. In this example, the application recommends a training session comprising three rounds of tapping sequences. The disclosed frustration sequence differs from existing tapping sequences targeting the frustration blockage by manipulating more acupuncture points, and by having unique sequences and combinations of points. With reference to FIG. 11, Round One includes a 15-point tapping sequence. With reference to FIG. 12, Round Two includes an 18-point tapping sequence derived from the 28 foundational points, and activates meridians associated with the following: liver, gall bladder, triple warmer, kidney, bladder, small intestine, heart, spleen, stomach, large intestine, lung, governing, yang linking, and ear relaxation point. Round Three uses the 15-point tapping sequence from Round One, and incorporates a grounding sequence followed by a shielding sequence at the end of the third round. Some versions may be executed under strict time limits, such as those imposed by social media applications. Mantras and affirmations maybe used in any of the three rounds. The three rounds of tapping sequences are summarized below.

Frustration and/or Anger Training Session

### Round #1

1) Small Intestine: SI-3 (Side of Hand)
2) Governing Vessel: GV-20 (Top of Head)
3) Bladder: B-2 (Eyebrow)
4) Gall Bladder: GB-1 (Side of Eye)
5) Stomach: S-2 (Under Eye)
6) Governing Vessel: GV-26 (Under Nose)
7) Central Vessel: CV-24 (Chin)
8) Kidney: KI-27 (Collarbone)
9) Spleen: SP-21 (Side of Rib)
10)Lung: LU-11 (Thumb)
11)Large Intestine: LI-1 (Index Finger)
12)Pericardium: P-9 (Middle Finger)
13)Triple Warmer: TW-1 (Ring Finger)
14)Heart: H-9 (Pinky Finger)
15)Triple Warmer: TW-3 (Gamut Point)

### Round #2

1) Small Intestine: SI-3 (Side of Hand)
2) Governing Vessel: GV-20 (Top of Head)
3) Ear Relaxation Point: Extra Point
4) Bladder: B-2 (Eyebrow)
5) Gall Bladder: GB-1 (Side of Eyebrows)
6) Stomach: S-2 (Under Eyes)
7) Governing Vessel: GV-26 (Under Nose)
8) Kidney: KI-27 (Collarbone)
9) Spleen: SP-21 (Side of Rib)
10)Liver: LIV-14 (Below Chest)
11)Stomach: S-36 (Outside of Knee)
12)Lung: LU-5 (Inner Elbow)
13)Yang Linking: SJ-5 (Top Mid Arm)
14)Heart: H-7 (Inside Bottom Wrist)
15)Large Intestine: LI-4 (Thumb—Index Web)
16)Lung: LU-11 (Thumb)
17)Heart: H-9 (Pinky)
18)Triple Warmer: TW-3 (Gamut)

### Round #3

1) Small Intestine: SI-3 (Side of Hand)
2) Governing Vessel: GV-20 (Top of Head)
3) Bladder: B-2 (Eyebrow)
4) Gall Bladder: GB-1 (Side of Eye)
5) Stomach: S-2 (Under Eye)
6) Governing Vessel: GV-26 (Under Nose)
7) Central Vessel: CV-24 (Chin)
8) Kidney: KI-27 (Collarbone)
9) Spleen: SP-21 (Side of Rib)
10)Lung: LU-11 (Thumb)
11)Large Intestine: LI-1 (Index Finger)
12)Pericardium: P-9 (Middle Finger)
13)Triple Warmer: TW-1 (Ring Finger)
14)Heart: H-9 (Pinky Finger)
15)Triple Warmer: TW-3 (Gamut Point)
16)Grounding Technique
17)Shielding Technique

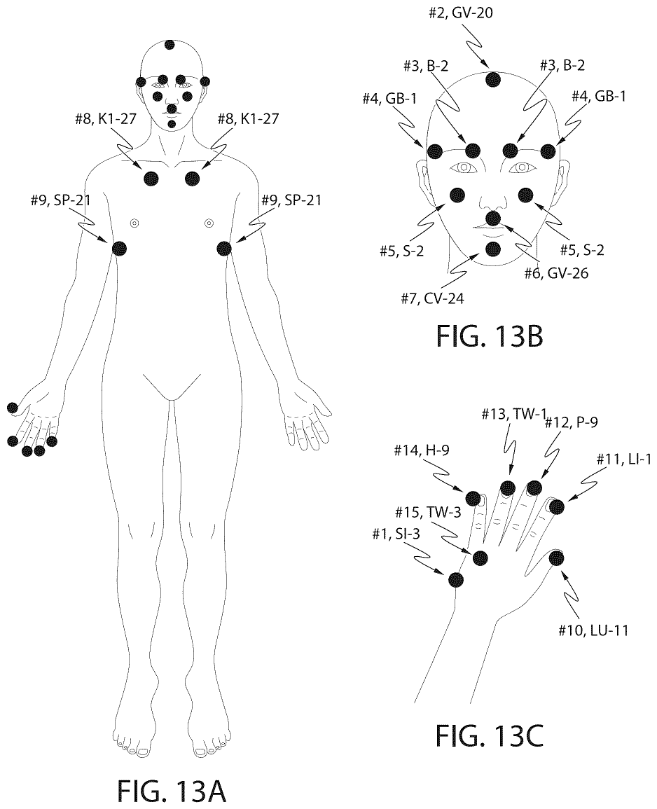

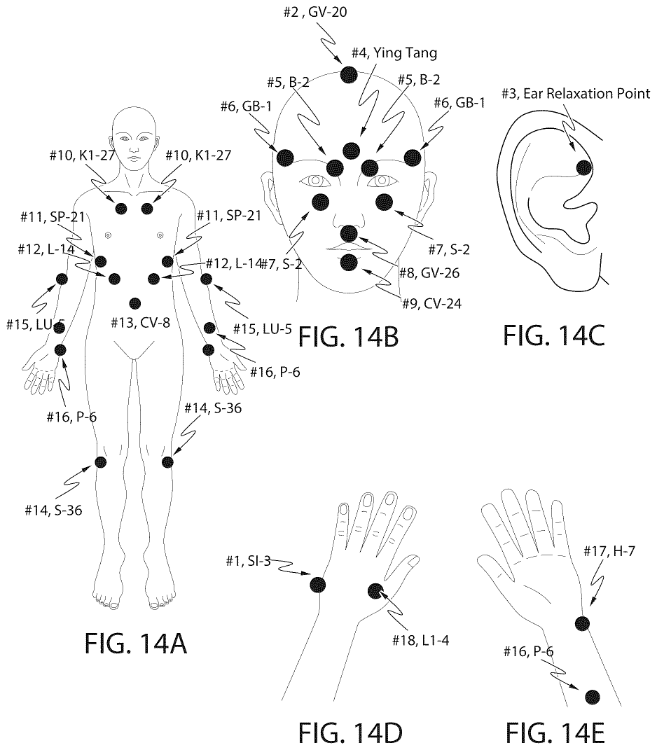

An example session of the disclosed invention targeting the blockage of lack of confidence and/or insecurity will now be described. In this example, the application recommends a training session comprising three rounds of tapping sequences. The disclosed confidence sequence differs from existing tapping sequences targeting the confidence blockage by manipulating more acupuncture points, and by having unique sequences and combinations of points. With reference to FIG. 13, Round One includes a 15-point tapping sequence. With reference to FIG. 14, Round Two includes a unique 18-point tapping sequence based upon the 28 foundational points, and activates meridians associated with the following: liver, gall bladder, kidney, bladder, small intestine, pericardium, heart, spleen, stomach, large intestine, lung, governing vessel, central vessel, yang linking, and the ear relaxation point. Round Three uses the 15-point tapping sequence from Round One, and incorporates a grounding sequence followed by a shielding sequence at the end of the third round. Some versions may be executed under strict time limits, such as those imposed by social media applications. Mantras and affirmations maybe used in any of the three rounds. The three rounds of tapping sequences are summarized below. Low Confidence and/or Insecurity Training Session Round #1
1) Small Intestine: SI-3 (Side of Hand)
2) Governing Vessel: GV-20 (Top of Head)
3) Bladder: B-2 (Eyebrows)
4) Gall Bladder: GB-1 (Side of Eye)
5) Stomach: S-2 (Under Eye)
6) Governing Vessel: GV-26 (Under Nose)
7) Central Vessel: CV-24 (Chin)
8) Kidney: KI-27 (Collarbone)
9) Spleen: SP-21 (Side of Rib)
10)Lung: LU-11 (Thumb Finger)
11)Large Intestine: L-1 (Index Finger)
12)Pericardium: P-9 (Middle Finger)
13)Triple Warmer: TW-1 (Ring Finger)
14)Heart: H-9 (Pinky)
15)Triple Warmer: TW-3 (Gamut)

### Round #2 Specific Confidence (Lack of Confidence)

1) Small Intestine: SI-3 (Side of Hand)
2) Governing Vessel: GV-20 (Top of Head)
3) Ear Relaxation Point
4) Ying Tang: Extra Point
5) Bladder: B-2 (Eyebrows)
6) Gall Bladder: GB-1 (Side of Eyes)
7) Stomach: S-2 (Under eyes)
8) Governing Vessel: GV-26 (Under nose)
9) Central Vessel: CV-24 (Chin)
10)Kidney: KI-27 (Collarbone points)
11)Spleen: SP-21 (Side of Ribs)
12)Liver: L-14 (Under Chest)
13)Central Vessel: CV-8 (Navel)
14)Stomach: S-36 (Side of knees)
15)Lung: LU-5 (Inside of elbow)
16)Pericardium: P-6 (Inside lower forearm)
17)Heart: H-7 (Inside bottom wrist)
18)Large Intestine: LI-4 (Webbing of Thumb)

### Round #3

1) Small Intestine: SI-3 (Side of Hand)
2) Governing Vessel: GV-20 (Top of Head)
3) Bladder: B-2 (Eyebrows)
4) Gall Bladder: GB-1 (Side of Eye)
5) Stomach: S-2 (Under Eye)
6) Governing Vessel: GV-26 (Under Nose)
7) Central Vessel: CV-24 (Chin)
8) Kidney: KI-27 (Collarbone)
9) Spleen: SP-21 (Side of Rib)
10)Lung: LU-11 (Thumb Finger)
11)Large Intestine: L-1 (Index Finger)
12)Pericardium: P-9 (Middle Finger)
13)Triple Warmer: TW-1 (Ring Finger)
14)Heart: H-9 (Pinky)
15)Triple Warmer: TW-3 (Gamut)
16)Grounding Technique
17)Shielding Technique

As detailed above, the disclosed invention may be used to target more common physical and emotional blockages (anxiety/stress, physical pain/discomfort, lack of focus, performance, frustration, confidence). A further application of the disclosed techniques tailors sequences to address more specific individualized emotions, states, physical issues, and performance issues. For example, the application may recommend a training session comprising three rounds of tapping sequences. Round One includes a 15-point tapping sequence. Round Two includes a unique tapping sequence based upon the 28 foundational points. Round Three uses a standard tapping sequence and incorporates a grounding sequence followed by a shielding sequence. All sequences may be executed under strict time limits. In other versions of the sequence, abbreviated tapping sequences may be used.

In addition to addressing specific conditions or issues experienced by the practitioner, the system also includes generally applicable sequences. An example session of the disclosed invention having a broad or general application will now be described. In this example, the application recommends a training session comprising three rounds of tapping sequences. Round One includes a 15-point tapping sequence. Round Two includes a unique tapping sequence based upon the 28 foundational points. Round Three uses a standard tapping sequence and may also incorporate mantras, grounding, and/or shielding sequences, all executed under strict time limits. In other versions of the general sequence, abbreviated tapping sequences may be used, and mantras, grounding, and/or shielding may be used as standalone techniques or as part of shorter combinations. The three rounds of tapping sequences for the General Training Session are summarized below.

### General Training Session

### Round 1:

1) Small Intestine: SI-3 (Side of Hand)
2) Governing Vessel: GV-20 (Top of Head)
3) Bladder: B-2 (Eyebrow)
4) Gall Bladder: GB-1 (Side of Eye)
5) Stomach: S-2 (Under Eye)
6) Governing Vessel: GV-26 (Under Nose)
7) Central Vessel: CV-24 (Chin)
8) Kidney: KI-27 (Collarbone)
9) Spleen: SP-21 (Side of Rib)
10)Lung: LU-11 (Thumb)
11)Large Intestine: LI-1 (Index Finger)
12)Pericardium: P-9 (Middle Finger)
13)Triple Warmer: TW-1 (Ring Finger)
14)Heart: H-9 (Pinky Finger)
15)Triple Warmer: TW-3 (Gamut Point)

### Round 2

1) Small Intestine: SI-3 (Side of Wrist)
2) Small Intestine: SI-4 (Side of Wrist)
3) Governing Vessel: GV-20 (Top of Head)
4) Ear: Relaxation Point (No Meridian): (Top of Ear)
5) Extra Point (No Meridian): Ying Tang Extra Point #1 (Between Eyebrows)
6) Bladder: B-2 (Eyebrow)
7) Gall Bladder: GB-14 (Above Middle of Eyebrow)
8) Gall Bladder: GB-1 (Side of Eye)
9) Stomach: S-2 (Under Eye)
10)Governing Vessel: GV-26 (Under Nose)
11)Central Vessel: CV-24 (Chin)
12)Kidney: KI-27 (Collarbone)
13)Spleen: SP-21 (Side of Rib)
14)Liver: LIV-14 (Under Chest)
15)Central Vessel: CV-8 (Navel)
16)Stomach: S-36 (Knee)
17)Lung: LU-5 (Inner Elbow)
18)Yang Wei Mai (Yang Linking): SJ-5 (Top Mid Arm)
19)Yin Wei Mai (Yin Linking): P-6 (Bottom Mid Arm)
20)Pericardium: P-7 (Middle Bottom Wrist)
21)Heart: H-7 (Outside Bottom Wrist)
22)Large Intestine: LI-4 (Thumb—Index Web)
23)Lung: LU-11 (Thumb)
24)Large Intestine: LI-1 (Index Finger)
25)Pericardium: P-9 (Middle Finger)
26)Triple Warmer: TW-1 (Ring Finger)
27)Heart: H-9 (Pinky)
28)Triple Warmer: TW-3 (Gamut)

### Round 3

1) Small Intestine: SI-3 (Side of Hand)
2) Governing Vessel: GV-20 (Top of Head)
3) Bladder: B-2 (Eye Brow)
4) Gall Bladder: GB-1 (Side of Eye)
5) Stomach: S-2 (Under Eye)
6) Governing Vessel: GV-26 (Under Nose)
7) Central Vessel: CV-24 (Chin)
8) Kidney: KI-27 (Collarbone)
9) Spleen: SP-21 (Side of Rib)
10)Lung: LU-11 (Thumb)
11)Large Intestine: LI-1 (Index Finger)
12)Pericardium: P-9 (Middle Finger)
13)Triple Warmer: TW-1 (Ring Finger)
14)Heart: H-9 (Pinky Finger)
15)Triple Warmer: TW-3 (Gamut Point)
16)Grounding Technique
17)Shielding Technique

An example session of disclosed invention addressing a perceived lack of energy experienced by the practitioner will now be described. In this example, the application recommends a training session comprising three rounds of tapping sequences. Round One includes a 15-point tapping sequence. Round Two includes a 17-point tapping sequence derived from the 28 foundational points, and activates meridians associated with the following: liver, gall bladder, kidney, bladder, small intestine, spleen, stomach, large intestine, lung, governing vessel, central vessel, triple warmer, and the ear relaxation point. Round Three uses the 15-point tapping sequence from Round One, and incorporates a grounding sequence followed by a shielding sequence at the end of the third round. Some versions may be executed under strict time limits, such as those imposed by social media applications. Mantras and affirmations maybe used in any of the three rounds. The three rounds of tapping sequences are summarized below.

### High Vibe Energy Increasing Training Session

### Round 1:

1) Small Intestine: SI-3 (Side of Hand)
2) Governing Vessel: GV-20 (Top of Head)
3) Bladder: B-2 (Eyebrow)
4) Gall Bladder: GB-1 (Side of Eye)
5) Stomach: S-2 (Under Eye)
6) Governing Vessel: GV-26 (Under Nose)
7) Central Vessel: CV-24 (Chin)
8) Kidney: KI-27 (Collarbone)
9) Spleen: SP-21 (Side of Rib)
10)Lung: LU-11 (Thumb)
11)Large Intestine: LI-1 (Index Finger)
12)Pericardium: P-9 (Middle Finger)
13)Triple Warmer: TW-1 (Ring Finger)
14)Heart: H-9 (Pinky Finger)
15)Triple Warmer: TW-3 (Gamut Point)

### Round 2:

1) Small Intestine: SI-3 (Side of Hand)
2) Small Intestine: SI-4 (Side of Wrist)
3) Governing Vessel: GV-20 (Top of Head)
4) Ear Relaxation Point
5) Bladder: B-2 (Eyebrows)
6) Gall Bladder: GB-1 (Side of Eyes)
7) Stomach: S-2 (Under eyes)
8) Governing Vessel: GV-26 (Under nose)
9) Central Vessel: CV-24 (Chin)
10)Kidney: KI-27 (Collarbone points)
11)Spleen: SP-21 (Side of Ribs)
12)Liver: L-14 (Under Chest)
13)Central Vessel: CV-8 (Navel)
14)Stomach: S-36 (Side of knees)
15)Lung: LU-5 (Inside of elbow
16)Large Intestine: LI-4 (Webbing of Thumb)
17)Triple Warmer: TW-3 (Gamut Point)

### Round 3:

1) Small Intestine: SI-3 (Side of Hand)
2) Governing Vessel: GV-20 (Top of Head)
3) Bladder: B-2 (Eyebrow)
4) Gall Bladder: GB-1 (Side of Eye)
5) Stomach: S-2 (Under Eye)
6) Governing Vessel: GV-26 (Under Nose)
7) Central Vessel: CV-24 (Chin)
8) Kidney: KI-27 (Collarbone)
9) Spleen: SP-21 (Side of Rib)
10)Lung: LU-11 (Thumb)
11)Large Intestine: LI-1 (Index Finger)
12)Pericardium: P-9 (Middle Finger)
13)Triple Warmer: TW-1 (Ring Finger)
14)Heart: H-9 (Pinky Finger)
15)Triple Warmer: TW-3 (Gamut Point)
16)grounding Technique
17)Shielding technique

### Special Use Protocols: SOS, Quick Hitters, & Boosters

Special Use Protocol include Quick Hitters, which are for practitioners to use right away in the moment to feel better emotionally or to improve performance. For example, during moments of distress, anxiety, pain, or frustration, practitioners can use a quick hitter sequence to boost confidence, raise their energy, to find calm, to focus, to relax, and to feel grounded. Booster sessions help practitioners improve longer term, i.e., not immediate challenges like public speaking poise, inconsistent performance, etc. SOS sequences are for acute distress, and help the practitioner address the situation by improving focus, energizing, creating an uplifted mood, and to optimize overall mood, energy, and performance. Each Special use sequence is comprised of one differentiated tapping protocol round drawn from the overall 28 master points, that can be cycled through repeatedly. Grounding techniques followed by shielding techniques will be included at the end of the last tapping round to reinforce, integrate, and amplify the sequence. Practitioners may also receive shorter abbreviated SOS tapping point sequences, drawn from a limited list of SOS points. These shorter sequences can be used within or away from the application.

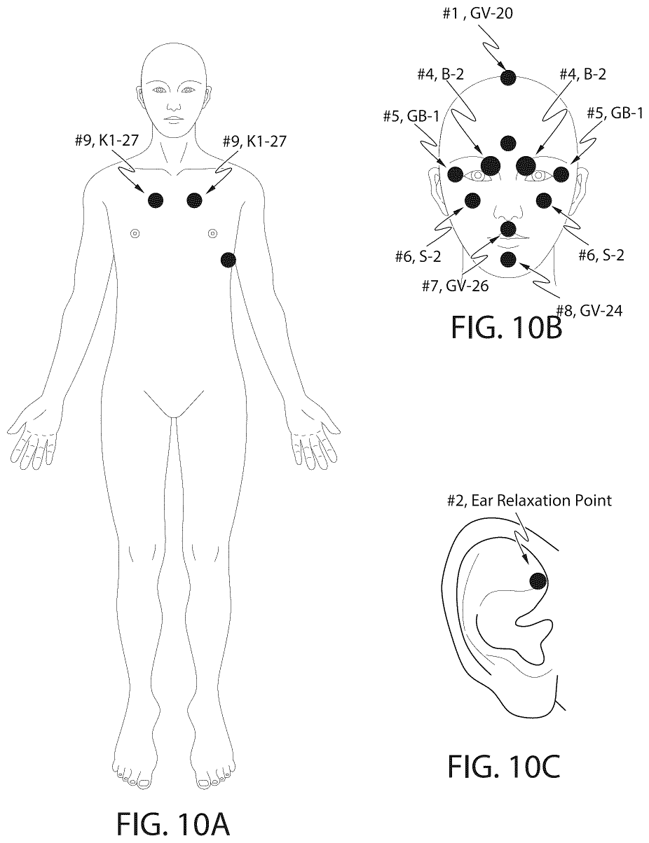

An example Special Use Protocol targeting blockages during a performance, and for optimizing focus will now be described. In this example, the application recommends a training session comprising one continuous round of tapping sequence. With reference to FIG. 10, the single round includes a 9-point tapping sequence, derived from the 28 foundational points, and activates meridians associated with the following: governing vessel, central vessel, gall bladder, kidney, bladder, stomach, and uses the extra ear relaxation point, and Ying Tang point between the eyebrows. After the tapping sequence, the practitioner executes a grounding sequence followed by a shielding sequence. Some versions may be executed under strict time limits, such as those imposed by social media applications. Mantras and affirmations maybe used in any of the three rounds. The Performance/Focus tapping sequence, with grounding and shielding techniques are summarized below.

### Performance/Focus Training Session

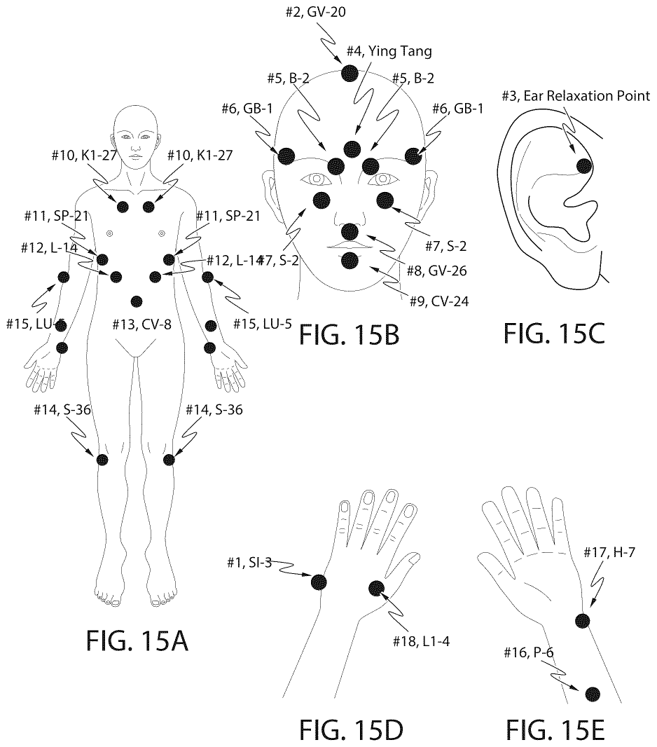

1) Governing Vessel: GV-20 (Top of Head)
2) Ear Relaxation Point
3) Ying Tang: (Between Eyebrows)
4) Bladder: B-2 (Eyebrow)
5) Gall Bladder: GB-1 (Side of Eye)
6) Stomach: S-2 (Under Eye)
7) Governing Vessel: GV-26 (Under Nose)
8) Central Vessel: CV-24 (Chin)
9) Kidney: KI-27 (Collarbone)
10)Grounding Technique
11)Shielding technique
With reference to FIG. 15, the SOS Confidence Booster includes a unique 18-point tapping sequence derived from the 28 foundational points. It activates meridians associated with the following: liver, gall bladder, pericardium, kidney, bladder, small intestine, heart, spleen, stomach, large intestine, lung, governing vessel, central vessel, Yin linking, and the ear relaxation point. Grounding and shielding techniques are included at the end of the last cycled round to reinforce, integrate, and amplify the tapping sequence.

### SOS Confidence Booster

1) Small Intestine: SI-3 (Side of Hand)
2) Governing Vessel: GV-20 (Top of Head)
3) Ear Relaxation Point
4) Ying Tang: Extra Point
5) Bladder: B-2 (Eyebrows)
6) Gall Bladder: GB-1 (Side of Eyes)
7) Stomach: S-2 (Under eyes)
8) Governing Vessel: GV-26 (Under nose)
9) Central Vessel: CV-24 (Chin)
10)Kidney: KI-27 (Collarbone points)
11)Spleen: SP-21 (Side of Ribs)
12)Liver: L-14 (Under Chest)
13)Central Vessel: CV-8 (Navel)
14)Stomach: S-36 (Side of knees)
15)Lung: LU-5 (Inside of elbow
16)Pericardium: P-6 (Inside lower forearm)
17)Heart: H-7 (Inside bottom wrist)
18)Large Intestine: LI-4 (Webbing of Thumb)
19)Grounding technique
20)Shielding Technique

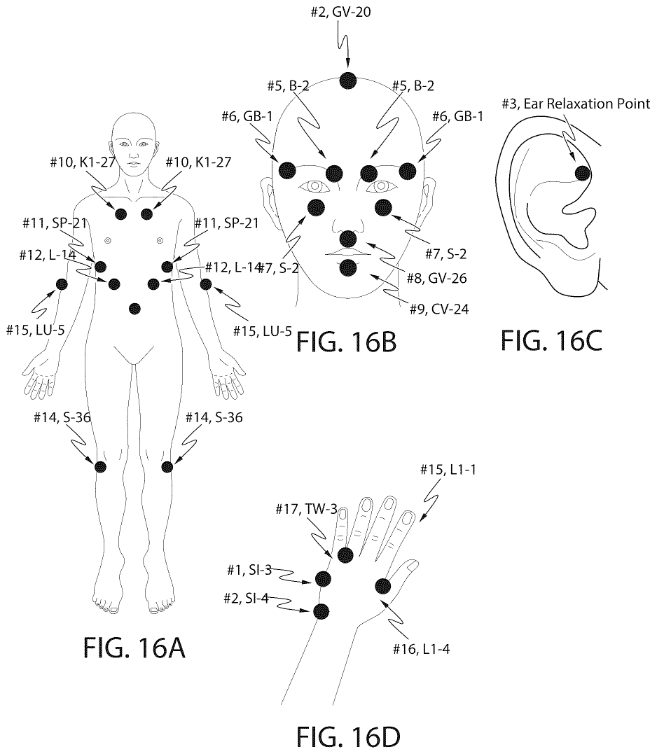

With reference to FIG. 16, the SOS High Vibe sequence is intended to raise perceived energy levels in the practitioner, and includes a unique 17-point tapping sequence based upon the 28 foundational points, and activates meridians associated with the following: liver, gall bladder, kidney, bladder, small intestine, spleen, stomach, large intestine, lung, governing vessel, central vessel, triple warmer, and the ear relaxation point. Grounding techniques followed by shielding techniques are included at the end of the last cycled round to reinforce, integrate, and amplify the tapping sequence.

### SOS High Vibe

1) Small Intestine: SI-3 (Side of Hand)
2) Small Intestine: SI-4 (Side of Wrist)
3) Governing Vessel: GV-20 (Top of Head)
4) Ear Relaxation Point
5) Bladder: B-2 (Eyebrows)
6) Gall Bladder: GB-1 (Side of Eyes)
7) Stomach: S-2 (Under eyes)
8) Governing Vessel: GV-26 (Under nose)
9) Central Vessel: CV-24 (Chin)
10)Kidney: KI-27 (Collarbone points)
11)Spleen: SP-21 (Side of Ribs)
12)Liver: L-14 (Under Chest)
13)Central Vessel: CV-8 (Navel)
14)Stomach: S-36 (Side of knees)
15)Lung: LU-5 (Inside of elbow
16)Large Intestine: LI-4 (Webbing of Thumb)
17)Triple Warmer: TW-3 (Gamut Point)
18)Grounding technique
19)Shielding technique

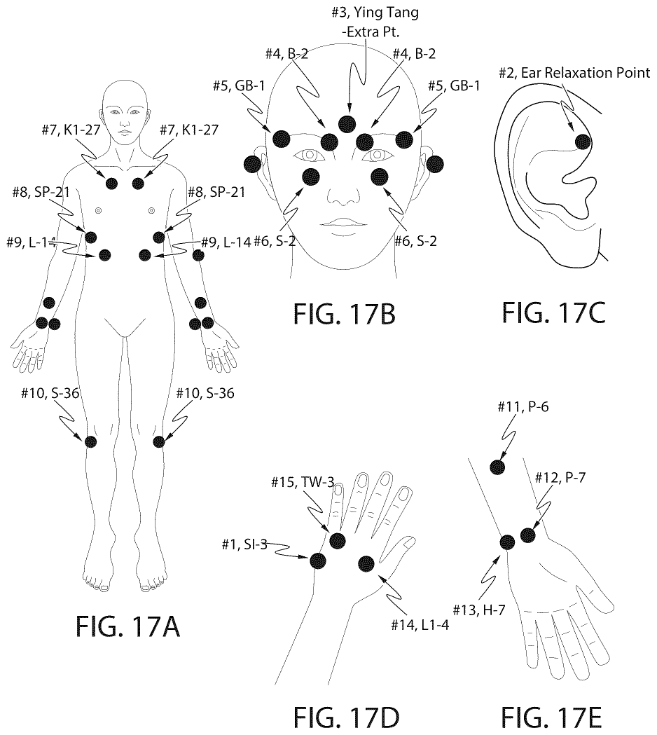

With reference to FIG. 17, the SOS Calm sequence includes a unique 15-point tapping sequence based upon the 28 foundational points, and activates meridians associated with the following: gall bladder, triple warmer, kidney, bladder, small intestine, spleen, stomach, large intestine, liver, pericardium, heart, Yin linking, ear relaxation point, and Ying Tang point. Grounding techniques followed by shielding techniques are included at the end of the last tapping round to reinforce, integrate, and amplify the tapping sequence.

### SOS Calm

1. Small Intestine: SI-3 (Side of Hand)
2. Ear: Relaxation Point (Top of Ear)
3. Extra Point: Ying Tang Extra Point #1 (Between Eyebrow)
4. Bladder: B-2 (Eyebrow)
5. Gall Bladder: GB-1 (Side of Eye)
6. Stomach: S-2 (Under Eye)
7. Kidney: KI-27 (Collarbone)
8. Spleen: SP-21 (Side of Rib)
9. Liver: LIV-14 (Under Chest)
10. Stomach: S-36 (Knee)
11.Yin Wei Mai (Yin Linking): P-6 (Bottom Mid Arm)
12. Pericardium: P-7 (Middle of Wrist)
13.Heart: H-7 (Outside Bottom Wrist)
14.Large Intestine: LI-4 (Thumb—Index Web)
15.Triple Warmer: TW-3 (Gamut)
16.Grounding Technique
17. Shielding Technique

With further reference to FIG. 12, the SOS Frustration & Anger sequence includes a unique 18-point tapping sequence based upon the 28 foundational points, and activates meridians associated with the following: liver, gall bladder, triple warmer, kidney, bladder, small intestine, heart, spleen, stomach, large intestine, lung, governing, yang linking, and ear relaxation point. Grounding techniques followed by shielding techniques are included at the end of the last cycled round to reinforce, integrate, and amplify the tapping sequence.

### SOS Frustration/Anger

1) Small Intestine: SI-3 (Side of Hand)
2) Governing Vessel: GV-20 (Top of Head)
3) Ear Relaxation Point
4) Bladder: B-2 (Eyebrows)
5) Gall Bladder: GB-1 (Side of Eyes)
6) Stomach: S-2 (Under eyes)
7) Governing Vessel: GV-26 (Under Nose)
8) Kidney: KI-27 (Collarbone points)
9) Spleen: SP-21 (Side of Ribs)
10)Liver: L-14 (Under Chest)
11)Stomach: S-36 (Side of knees)
12)Lung: LU-5 (Side of Elbow)
13)San Jiao: SJ-5 (Middle forearm)
14)Heart: H-7 (Inside bottom wrist)
15)Large Intestine: LI-4 (Webbing of Thumb)
16)Lung: LU-11 (Thumb)
17)Heart: H-9 (Pinky)
18)Triple Warmer: TW-3 (Gamut)
19)Grounding Technique
20)Shielding Technique

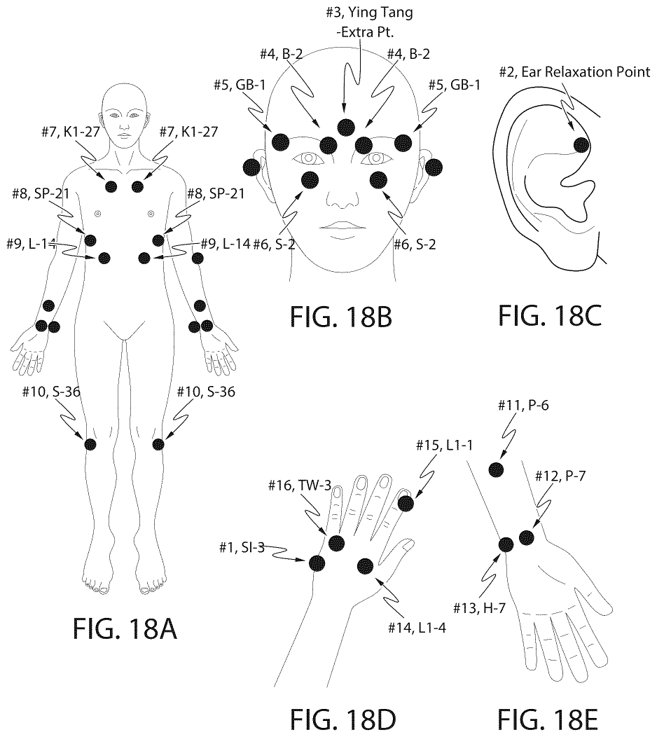

With reference to FIG. 18, the SOS Physical Pain/Discomfort sequence includes a unique 16-point tapping sequence based upon the 28 foundational points, and activates meridians associated with the following: gall bladder, triple warmer, kidney, bladder, small intestine, spleen, stomach, large intestine, liver, pericardium, heart, Yin linking, ear relaxation point, and Ying Tang point. Grounding techniques followed by shielding techniques are included at the end of the last cycled round to reinforce, integrate, and amplify the tapping sequence.

### SOS Physical Pain

1) Small Intestine: SI-3 (Side of Hand)
2) Ear Relaxation Point
3) Ying Tang: Extra Point
4) Bladder: B-2 (Eyebrows)
5) Gall Bladder: GB-1 (Side of Eyes)
6) Stomach: S-2 (Under eyes)
7) Kidney: KI-27 (Collarbone points)
8) Spleen: SP-21 (Side of Ribs)
9) Liver: L-14 (Under Chest)
10)Stomach: S-36 (Side of knees)
11)Pericardium: P-6 (Inside lower forearm)
12)Pericardium: P-7 (Wrist)
13)Heart: H-7 (Inside bottom wrist)
14)Large Intestine: LI-4 (Webbing of Thumb)
15)Large Intestine: LI-1 (Index Finger)
16)Triple Warmer: TW-3 (Gamut)
17)Grounding Technique
18)Shielding Technique

On-ramping/on-boarding and other abbreviated sequences. The disclosed invention also includes on-ramping or on-boarding sequences which are shorter subsets of tapping points drawn from the 28 foundational points aimed at education and acclimating users to the longer rounds of tapping.

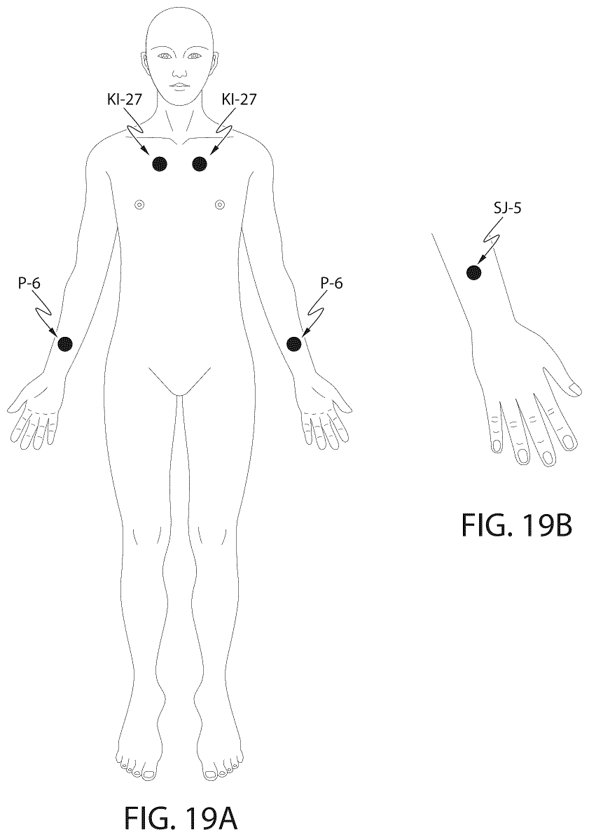

With reference to FIG. 19, In-Performance abbreviated tapping sequences designed to be performed during a game or performance are described. These abbreviated sequences are drawn from the 28 foundational points and can be used as take-away sequences, and may be used with or without guidance from the application. These may be used as part of longer sessions, SOS's, or may also be used as stand-alone trainings. The acupuncture points found to be most effective for In-Performance tapping are the following: (under eye) S-2, (collarbone) KI-27, (thumb) LU-11, (index finger) LI-1, (side of hand) SI-3, (middle finger) P-9, (gamut) TW-3, (pinky) H-9, (under eye) S-2, (knee) S-36 and the (mid arm) P-6 on the Yin linking, and (mid arm) SJ-5 on the Yang linking meridians. Four examples of in-performance tapping combinations follow below:

### In-Performance Tapping Sequences

1) Collarbone KI-27
2) Collarbone KI-27 and Mid-Arm P-6
3) Collarbone KI-27 and Mid-Arm SJ-5
4) Collarbone KI-27, Mid-Arm SJ-5, and P-6

The application may recommend that the practitioner execute a standard In-Performance tapping sequence at various times during a basketball game. For example, prior to a basketball game, the application recommends that if the practitioner experiences a reduction in emotional energy, an increase in negative emotion, or a drop in performance during the game, the practitioner should execute an In-Performance tapping sequence within a set time limit. When the practitioner experiences one of the triggering conditions, the practitioner then manipulates a sequence of acupuncture points. The practitioner may also perform one or more mantras audibly and/or inaudibly, in coordination with the tapping sequence. In order to optimize performance by the practitioner, the application may recommend execution of In-Performance tapping sequences and accompanying modalities throughout the game, during warmups, before the start of the game, during timeouts, during half-time or other longer breaks, and/or after mistakes, missed shots, dropped passes, etc. In another example, an entrepreneur may use In-Performance Tapping techniques to improve their performance of a presentation by executing the sequence immediately prior to the presentation, and/or at weak points or mistakes that occur during the presentation. Or the practitioner may perform the sequence pre-emptively and periodically during the presentation. Any In-Performance tapping sequence may be accomplished with eyes open or closed, but must be accomplished within the time limit designated by the application.

The standard In-Performance tapping sequence may also be modified for specific in-performance situations and contexts. For example, a foul-line tapping sequence may be used to improve a basketball player's performance shooting foul shots. The foul-line tapping sequence may also be used by other athletes and performers prior to completing in-performance skills. The practitioner may tailor the sequence to his or her needs, such as by executing the sequence with eyes open or closed, performing the tapping sequence as a stand-alone discipline, or by reciting a coordinated mantra audibly or inaudibly, or other combination of modalities. With further reference to FIG. 19, an example foul-line tapping session is described below:

### Foul-Line Tapping Session

(1) Practitioner approaches free-throw line
(2) Practitioner manipulates SJ-5 10 times to neutralize emotional or behavioral residue of a missed-shot
(3) Practitioner executes first free throw attempt
(4) Practitioner manipulates SJ-5 5-10 times, then manipulates KI-27 5-10 times. Practitioner may also repeat a mantra audible and/or inaudibly, in-concert with specifically tapping one or more points, 5-25 times
(5) Practitioner completes the sequence within the foul shot limit of 10 seconds.
(6) The points include but are not limited to the 28 foundational points.

### Grounding and Shielding Techniques

In addition to tapping sequences and techniques, embodiments of the disclosed invention may use a grounding modality. For use outside the performance context, such as upon waking, throughout the day, pre-performance, and/or post-performance, the application may recommend a Long Grounding sequence. The Long Grounding sequences described herein provide the practitioner a prolonged amplification of energetic, emotional, and behavioral centering and integration. Grounding sequences and techniques create an internal emotional environment where the practitioner can harness, integrate, and anchor the emotional or energetic changes made through execution of other system modalities. Long grounding may be performed with eyes open or closed and is designed to be independent of stimuli external to the practitioner. In contrast to existing grounding techniques, the disclosed method may be used with tapping sequences or other modalities, is designed to be integrated with the practitioner's broader performance processes and may be completed within recommended time constraints. An example long grounding sequence is summarized below:

### Long Grounding Sequence:

(1) Practitioner secures the soles of their feet to the ground;
(2) Practitioner takes 5-25 deep inhalations through their nose and/or mouth;
(3) Practitioner envisions cords from the soles of the feet extending geographically around the earth north and south, and connecting together on the other side of the earth;
(4) Practitioner envisions cords from the soles of the feet extending directly through earth's layers until reaching earth's core;
(5) Practitioner feels their best performance moment, most peaceful moment, most relaxed moment, etc. ;
(6) Practitioner sustains feeling their best performance moment while bringing emotional energy back up from the earth and through their body;
(7) Practitioner completes the sequence within the required time frame.

The disclosed invention also includes In-Performance Grounding, an abbreviated in-performance version of the grounding modality. For example, a practitioner playing basketball may use an In-Performance grounding sequence to create the desired emotional or energetic environment prior to shooting free throws, during time-outs, at half-time, and/or after missed shots or mistakes. An example In-Performance grounding sequence is summarized below:

### In-Performance Grounding Sequence:

1) Before, during, or after performance, practitioner secures the soles of their feet to the ground;
2) Practitioner takes 3-10 deep inhalations through their nose and/or mouth;
3) Practitioner envisions cords from the soles of the feet extending directly through earth's layers until reaching earth's core;
4) Practitioner feels their best performance moment, most peaceful moment, most relaxed moment, etc. ;
5) Practitioner sustains feeling their best performance moment while bringing emotional energy back up from the earth and through their body;
6) Practitioner completes the sequence within the required time frame.

Embodiments of the disclosed invention may also use a shielding modality. Shielding creates an energetic buffer, or impervious energetic field, around the practitioner. Shielding orients the practitioner to envision an impenetrable energy field encircling the area surrounding the practitioner's physical body. Negative outside influences have long been shown to promote emotional discord, cause physical pain, and to impede performance. For example, a basketball player attempting free throws in front of 20,000 opposing fans will experience a great deal of hostility and negative energy directed toward them. Such negative energy can trigger undesirable emotions, instability, behavioral changes, and physical sensations—all of which can degrade performance. Shielding therefore provides the practitioner a tool to offset that external interference by harnessing the human energy system in a way that buffers outside distractions. As with the other modalities described herein, shielding can be used as a stand-alone technique, or may be used in-concert with tapping, grounding, mantras, or other modalities and inputs. Shielding may take the form of an extended version, the Long Shield, which is completed within 1-3 minutes, and an abbreviated version, the In-Performance Shield, which is executed in 3-59 seconds. An example long shield sequence and an example In-Performance shield sequence are summarized below:

### Long Shielding Sequence:

(1) A time is selected upon waking, before performances and competition, pre-performance, and/or throughout the day
(2) Practitioner selects the energetic interference they are experiencing
(3) Practitioner encircles their body with a buffer comprised of a positive energetic/emotional field
(4) Must be completed in the recommended time frame.

### In-Performance Shielding Sequence:

(1) A time is selected upon waking, before games and competition, pre-practice, and/or throughout the day
(2) Practitioner selects the energetic interference they are experiencing
(3) Practitioner encircles their body with a buffer comprised of a positive energetic/emotional field
(4)Must be completed in the recommended abbreviated time frame.

### Other Training Modalities

Embodiments of the disclosed invention may also use a mantra or affirmation modality. Mantras and affirmations may be used in conjunction with tapping sequences to reprogram positive behavioral changes through the use of affirmative statements. Mantras or affirmations include statements that tend to reinforce positive emotions, behavior, physical sensations, or performance. Mantras intersect and target specific emotions, mental states, and behaviors, directly corresponding to the meridian system, to strengthen and amplify improvement. For example, anger is associated with the liver meridian. Therefore, a mantra targeting the liver will be inversely related to, and will tend to neutralize, the negative emotion of anger, while promoting corresponding positive emotions or behaviors. Existing techniques using mantras do not correlate mantras to specific meridians, are not used with disclosed tapping sequences, and are not time constrained. Mantras may be executed as a stand-alone technique, or coordinated with other modalities. An example mantra sequence is described below:

### Mantra Sequence:

(1) Practitioner creates or selects an “I Am” statement, or another statement that reinforces the intention to be amplified. (e.g., “I am powerfully performing at the foul line.”).
(2) Practitioner repeats the mantra audibly or inaudibly
(3) Practitioner repeats the mantra in conjunction with previously identified tapping points (4)Mantra may be completed in designated time limit.

Embodiments of the disclosed invention also use color therapy to achieve performance improvement. With further reference to FIG. 3, colors are associated with each of the body's specific energy centers, or chakras, of the human energy system. Specific colors also intersect, and/or are associated with emotions, behavior, and meridians. When different colors are applied in a prescribed sequence, color therapy can work to neutralize energetic and emotional disruption within the human energy system. It has long been known that hospitals frequently paint their interiors pale green due to the healing properties associated of the color. McDonald's uses the colors red and yellow in their marketing campaigns because those colors are shown to increase the perception of hunger. Color therapy will be used to amplify improvement: (1) Intersecting with and neutralizing emotions, behavior, physical sensation and/or performance impediments; (2) once the user identifies the focus of work, or the energy/emotion and/or behavior to be adjusted, chakra colors will be used to support the practitioner's progress; (3) as the user progresses through the levels of inquiry and a tapping sequence is generated, color(s) will appear and positively affect corresponding meridians. For example, green is associated with healing and the fourth chakra, commonly known as the heart. If a heart-related emotion is identified through inquiry, the color green will be visually transmitted to the practitioner via a wearable, or through other means.

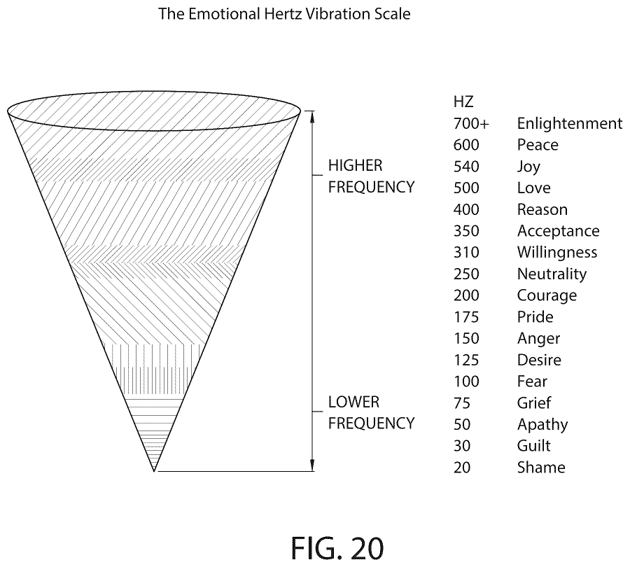

Embodiments of the disclosed invention also apply audible or in-audible sound waves, either to the ear or directly to the body, to achieve performance improvement. The human body is comprised of cells and organs that vibrate each at their own specific frequency. Audible and inaudible sonic frequencies can influence matter, physiology, and emotional/behavioral blockages. For example, opera singers can shatter glass with the right tone, and individuals exposed to classical music over a certain duration of time have displayed enhanced emotional intelligence. With reference to FIG. 20, specific frequencies can also influence emotional states and behaviors. Certain frequencies known as Schumann frequencies capture the interaction among the earth's magnetic fields and the resonant or effective frequencies associated organs, meridians, and/or emotional states of the human body. For example, fear has been shown to vibrate/be measured at 100 hertz. If the emotion of fear is identified through inquiry, the neutralizing auditory frequency, or range of frequencies, may be transmitted to neutralize the negative emotion. Applying a specific frequency and/or a range of frequencies to the human energy systems(s) neutralizes energetic/emotional blockages. This is especially true when used in-concert with the aforementioned modalities. Auditory tracks and/or programs may be based on time specific needs of athletes and performers, to support practice and in-performance optimization. Audio tracks and their frequency component are generated automatically based upon users'selections at prior levels of inquiry. Sound waves may used in at least four ways to amplify improvement: (1) to leverage the interrelationship between specific sonic frequencies and frequency ranges to provide the user with audible and inaudible sound waves that neutralize energetic/emotional and behavioral imbalances; (2) used as a sound wave component in-concert with the suite of aforementioned modalities: (3) customized frequencies are generated for the user, as the user progresses through the pathway/session; (4) post-session use to enhance in-session results.

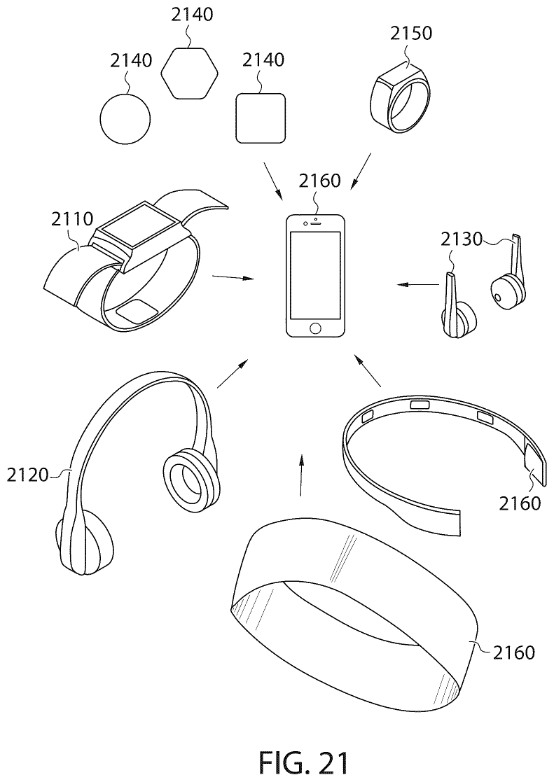

Embodiments of the disclosed invention may use one or more wearable devices to monitor progress through biofeedback, to allow practitioner interaction with the application, or to administer frequencies, vibrations, colors, or other inputs to the practitioner. With reference to FIG. 21, wearable device(s) may include a mechanism, and/or one or more sensors, and can be mounted on or in, for example, a Bluetooth, near-field communication (NFC), and/or wireless-enabled wrist watch or wearable 2110, wrist band, headphones 2120, earbuds/earphones 2130, adhesive patches 2140, ring 2150, sweatbands/headbands 2160, and/or can be present in or on other devices usable with the present invention, e.g., smartphones 2170 such as, the Apple iPhone, android device, or activity tracker such as Fitbit, or may be a uniquely designed wearable.

The wearable and/or corded conductor/transducer/adaptor/sensor will infuse, transduce and/or administer frequency and/or vibration to the body to neutralize emotional/energetic and behavioral impediments and improve performance. Modalities are included but are not limited to the methods listed below. The on-screen application deploys customized pathway/sessions, or stand-alone prescription methods, in-concert with frequency/vibration deployment via a wearable. For example, as above, if the emotion of fear has been identified through inquiry, the neutralizing frequency transmission(s), or range, will be transmitted directly into the body, to neutralize the negative emotion. The methods below will be used when the phone/wearable application is accessed in-concert with the aforementioned modalities. In addition, after closure of the app, during the daily routine and/or sleep, the wearable may continue to transmit frequency and vibration into the body. It may also be used in-concert with auditory frequency methods to amplify improvement. This portion of the wearable may be in the form of a strap, and/or band that attaches to a wired conductor, wrist watch, bracelet, ring, patch(s), headphones, headband, or device placed along specific energy meridians of the body.

Specific frequencies are transmitted into the body after levels of inquiry have indicated 1) emotional, 2) behavioral, 3) performance, or 4) chronic injury/physical needs. Energy, light, sound, and color are vibrations. This modality transduces digital audio, and/or specific and/or a range of hertz frequencies into the human energy system by way of a wearable/conductor. Digital audio, instead of being transmitted into auditory sound like one would do for music with headphones, is transduced into inaudible sensory vibration. This has been shown to instantaneously impact/interface with the human energy systems(s). These vibration(s) are sent and/or transmitted through the user's skin via a wearable/conductor. There are frequency-based modes and/or programs that will work in-concert with the other multi-layered modalities to amplify improvement. Frequency infuses the human energy systems(s) with waves that neutralize emotional/energetic and behavioral blockages. This method functions in at least three ways: (1) Specific frequencies/or frequency ranges, are transmitted through a wearable/conductor, remote, and/or a corded device; (2) the wearable/conductor transduces and transmits the digital audio in the form of energy, light, sound and color into frequencies that are transmitted into the human body; (3) the frequency(s) interfaces with the user's human energy system(s) to neutralize energetic/emotional and behavioral blockages and reduced pain. The user experience is potentially but not necessarily administered as per the following example:
(1) Practitioner moves through levels of inquiry, or selects desired frequency
(2) A Customized pathway/session is generated
(3) The practitioner's energy systems are vibrating at, and emitting, a less than optimal frequency which is the basis for decreased performance
(4) A frequency or range of frequencies based upon prior practitioner inquiry responses will be transduced by the conductor, impacting the practitioner's energy systems, to elevate frequency and positively impact performance
(5) Frequency can be used as a stand alone or in-concert with a customized session, and the aforementioned modalities.

Biofeedback Tracking: The wearable(s) may include a bio-metric and energetic-metric tracking function. The device may be in the form of, including, but not limited to a: watch, watch strap/band, ring, earpiece and/or a head phone device, bracelet, and/or adhesive patch(es) in existence or uniquely designed to work in conjunction with the application. The wearable may also be a wired device that connects into the phone and/or watch and can be held, placed, or attached to the user's body or clothes/shoes. The wearable will track physiological data, including but not limited to, heart rate, heart rate variability, respiratory variability, (pulse-CO₂) data, which is useful for showing a user's oxygen saturation (SO˜) galvanic skin response, temperature, facial coding, subtle energy waves, such as energy fields of the body, changes in brain waves, changes in frequencies in the body's energy field due to vacillations in emotions, behavior and individual energetics, by way of electrocardiography (EKG or ECG), electroencephalography (EEG), electromyography EMG), and/or a electrooculography (EOG). The wearable may capture, collect, and provide on-going biofeedback data to the app and/or on-line integration/cloud. This data would be tracked and used in the form of graphs, charts, and as a part of individual and collective ratings/rankings. The tracking will function as follows; (1) it captures biofeedback: (2) it captures energetic feedback; (3) it shares information with the mobile application, wearable tracking device and on-line storage/integration; (4) its effectiveness lies in its in-concert use.

The disclosed invention my also include additional elements discussed below.

Ratings and Tracking: Prior to, and following a timed training/session, practitioners will manually rate and input their energetic, emotional, and/or behavioral perceptions by way of a scaled rating. This data will be captured and presented back to the practitioner in the form of, including but not limited to, graphs, diagrams, figures, numbers, comparisons, charts of the progress, forecasts, and/or recommendations. The practitioner may also input/select ratings at other stages of the process as well. Biofeedback and tracking may occur before, during and after the session, and may be tracked inter-day, and intra-day, being sent back to the mobile application/on-line/cloud/storage

On-line Integration/Cloud/Physical/Storage: This invention may capture practitioner data/information for various purposes, including but not limited to: Biofeedback, energetic biofeedback, future recommendations, development, individual tracking, community tracking and social media/boards. Cloud/storage may communicate with wearable app, and phone app. As baselines are recorded and collected, the software will generate comparisons of manual, biometric and energetic levels. This may or may not be compiled into an overall score. This will give the practitioner a sense of progress.

Post-Session Recommendations: Once a practitioner has completed a session, recommendations for increasing the benefit of session results may be sent by way of an audible frequency, audio/visual recordings, wearable transmitted frequency, mantras, grounding, and shielding, and/or abbreviated tapping sequences, e.g., SOS Sequences. Recommendations may include, but are not limited to mobile application notifications and wearable notifications.

Progress Charting: The manual, biometric and energetic ratings and completion requirements will be measured against individual progress, and community progress.

Leaderboards: Will be used generally to illustrate practitioner progress including, but not limited to, interactive graphics, charts and diagrams which illustrate participation, volume completion, improvement and gains across blockage reduction, emotional intelligence, and performance.

Community Interaction: Social Media: The software will allow practitioners to share their tracking progress via social media, including but not limited to, Instagram, Facebook, anecdotal sharing, and Twitter etc. Community Discussion Boards: In-application boards will allow practitioners to pose questions, provide comments, like and/or rate experiences and outcomes. Practitioners will be rewarded for having attained various thresholds reached, including but not limited to, participation, improvement(s), comments and contributions. For example, a point system may exist where practitioners acquire likes, comments and/or/points based on progress, improvement and/or contribution.

Although several processes have been disclosed herein as software, it may be appreciated by one of skill in the art that the same processes, functions, etc. may be performed via hardware or a combination of hardware and software. Similarly, although the present invention has been depicted as a hardwired system, these concepts may be applied to wireless systems and hybrid hardwired and wireless systems without departing from the scope of the present invention.

As will be understood by those familiar with the art, the disclosed invention may be embodied in other specific forms without departing from the spirit or essential characteristics thereof. Likewise, the particular naming and division of the modules, managers, functions, systems, layers, features, attributes, methodologies, and other aspects are not mandatory or significant, and the mechanisms that implement the invention or its features may have different names, divisions, and/or formats. Accordingly, the disclosure of the present invention is intended to be illustrative, but not limiting, of the scope of the invention.

### Summary of Claims

1. A method for improving human performance in time-constrained activities, the method comprising:
creating a profile for a practitioner, wherein the profile includes information relevant to an identity of the practitioner;
performing an inquiry of the practitioner, wherein the inquiry includes a plurality of questions relevant to one or more of an activity to be performed, a mental state of the practitioner, a physical state of the practitioner, and a performance blockage of the practitioner;
selecting a training session for the practitioner, wherein the training session includes one or more of a tapping sequence, a shielding sequence, and a grounding sequence;
communicating the training session requirements to the practitioner; and
guiding the practitioner during execution of the training session.
2. The method of claim 1, further comprising
collecting feedback from the practitioner after the training session ends; and
adding training session information and the feedback to the profile.
3. The method of claim 1, wherein the tapping sequence includes activating a subset of the following points: SI-3; SI-4; GV-20; Ear Relaxation Point; Ying Tang Extra Point; B-2; GB-14; GB-1; S-2; GV-26; CV-24; KI-27; SP-21; LIV-14; CV-8; S-36; LU-5; SJ-5; P-6; P-7; H-7; LI-4; LU-11; LI-1; P-9; TW-1; H-9; TW-3.
4. The method of claim 1, wherein the training session is a session protocol comprising the following sequential stages: a first round of tapping, a second round of tapping, a third round of tapping, a grounding sequence, and a shielding sequence, and wherein the first round and third round are the same.
5. The method of claim 1, wherein the training session is a special use protocol, comprising a single tapping sequence, a grounding sequence, and a shielding sequence.
6. The method of claim 5, wherein the practitioner executes the single tapping sequence a plurality of times prior to executing the grounding sequence and the shielding sequence.
7. The method of claim 1, further comprising a primer statement, wherein the practitioner executes the primer statement during execution of the tapping sequence.
8. The method of claim 1, further comprising a mantra wherein the practitioner executes the mantra during execution of the tapping sequence.
9. The method of claim 1, wherein the training session is configured to address one of the following performance blockages: stress and/or anxiety, physical pain and/or discomfort, frustration and/or anger, low confidence and/or insecurity, lack of focus, lack of energy, or a general blockage.
10. The method of claim 5, wherein the training session is configured to clear an in-performance blockage.
1. A system for improving human performance in time-constrained activities, the system comprising:
a computer configured for use by a practitioner to conduct a training session, the computer comprising a processor, processor associated memory, a user interface, and data storage, and wherein the processor, memory, interface, and data storage are configured to run an application on the computer for the training session, wherein the application is configured to exchange information with the practitioner via interaction with the interface, the application controlling the interface by presenting outputs on a display and reading data from the interface in relation to the outputs;
a library including a plurality of training sequences, wherein each of the plurality of training sequences addresses a performance blockage, and wherein the library is accessible by the application;
a user file for the practitioner, wherein the user file is configured to store user information about the practitioner, and wherein the user file is accessible by the application; and
wherein the application is configured to perform the following:
collect user information about the practitioner and store the user information in the user file, the user information including one or more of identity information, and training information,
select a training session from the plurality of training sequences based on the user information, and
guide the practitioner through the training session.
2. The system of claim 1, wherein the identity information includes information about the practitioner's identity, and wherein the training information includes information about a blockage or an activity performed by the practitioner.
3. The system of claim 1, wherein the identity information includes whether the practitioner is a new user or an existing user, and wherein the user information further includes feedback information, wherein the feedback information includes whether the training session was completed by the practitioner, and whether the blockage was improved.
4. The system of claim 3, wherein the application is further configured to
collect feedback information after the practitioner is guided through the training session, and
update the user file with the feedback information.
5. The system of claim 1, wherein the training session includes one or more of a tapping sequence, a shielding sequence, a grounding sequence, a primer, and a mantra.
6. The system of claim 1, wherein the training session includes a tapping sequence, and wherein the tapping sequence includes activating a subset of the following points: SI-3; SI-4; GV-20; Ear Relaxation Point; Ying Tang Extra Point; B-2; GB-14; GB-1; S-2; GV-26; CV-24; KI-27; SP-21; LIV-14; CV-8; S-36; LU-5; SJ-5; P-6; P-7; H-7; LI-4; LU-11; LI-1; P-9; TW-1; H-9; TW-3.
7. The system of claim 1, wherein the performance blockage is one of the following: a stress and/or anxiety blockage, a physical pain and/or discomfort blockage, a frustration and/or anger blockage, a low confidence and/or insecurity blockage, a lack of focus blockage, a low energy blockage, or a general blockage.
8. The system of claim 1, wherein the training session is one of the following: a session protocol, or a special use protocol.
9. The system of claim 8, wherein the training session is a session protocol comprising the following sequential stages: a first round of tapping, a second round of tapping, a third round of tapping, a grounding sequence, and a shielding sequence, and wherein the first round and third round are the same.
10. The system of claim 8, wherein the training session is a special use protocol, comprising a single tapping sequence, a grounding sequence, and a shielding sequence.
11. The system of claim 10, wherein the training session is configured to clear an in-performance blockage.

### Performance Improvement Systems Using Artificial Intelligence and/or Machine Learning

Some embodiments of the disclosed invention use AI/ML models to improve user intake, provide customized training sessions, and communicate customized training sessions to users. AI/ML models are trained using databases that are populated by data generated through expert trainers'interaction with trainees. By use of such AI/ML models, the system is able to provide scalable, expert quality training sessions that are customized for the user, and further that evolve with the user as training progresses based on user performance.

The disclosed training system takes a human performance enhancement program administered in-person by a trained subject matter expert and translates it for use through an AI-enhanced software application. Such translation is sorely needed. As an initial matter, there are few therapists with the skill set to coach performance improvement training. Coaches in the field require a substantial amount of training and years of experience to be able to effectively perform these tasks. In addition, in-person performance training may take 6 months to a year, and cost up to $10,000 before improvement is evident.

Translation of in-person training to a software application is not a trivial undertaking. Performing a training program under the supervision of a coach benefits from the coach's continuous instruction to the trainee to perform a particular tapping, grounding, or shielding sequence, the coach's ongoing assessment of the trainee's execution of the instructed sequence, and the coach's observation of the trainee's subsequent performance.

Another key component to the in-person performance program is the use of energy psychology to unblock a trainee's performance. A blockage typically forms when an individual experiences a failure in a time-sensitive activity and as a result experiences negative emotion. Such emotions may emerge during a subsequent activity, manifesting as a blockage that interferes with their performance of that subsequent activity. Performance enhancement training seeks to clear the blockage by prompting the trainee to remember the blocking emotion while preforming the instructed training sequence. Recalling the negative emotion during the training is thus critical to the effectiveness of the program. However, prompting the negative emotion is dependent on a trained coach eliciting the emotion at the correct time.

For example, assume a professional basketball player had a difficult game and missed several layups and shot a low free-throw percentage on the way to a team loss. A performance improvement coach tasked with developing a training program for the athlete would ask a sequence of onboarding questions to determine the best training protocol for the athlete. One aspect of the onboarding process is to determine the athlete's emotional state during the performance, and why they felt that emotion. The task of eliciting accurate emotions and potential underlying blockages is not a straightforward process and requires subject matter experts (SME) experience to perform.

The foregoing features of an in-person performance enhancement system make translation to a software application extremely difficult. The disclosed invention overcomes these difficulties at least in part by incorporating over 20 years of experience gained by an SME into AI/ML models that are used to create customized and curated training sessions for users. The result is a customizable training system that allows rapid onset improvement for lower cost, and without requiring supervision by a highly trained coach.

### Onboarding Engine

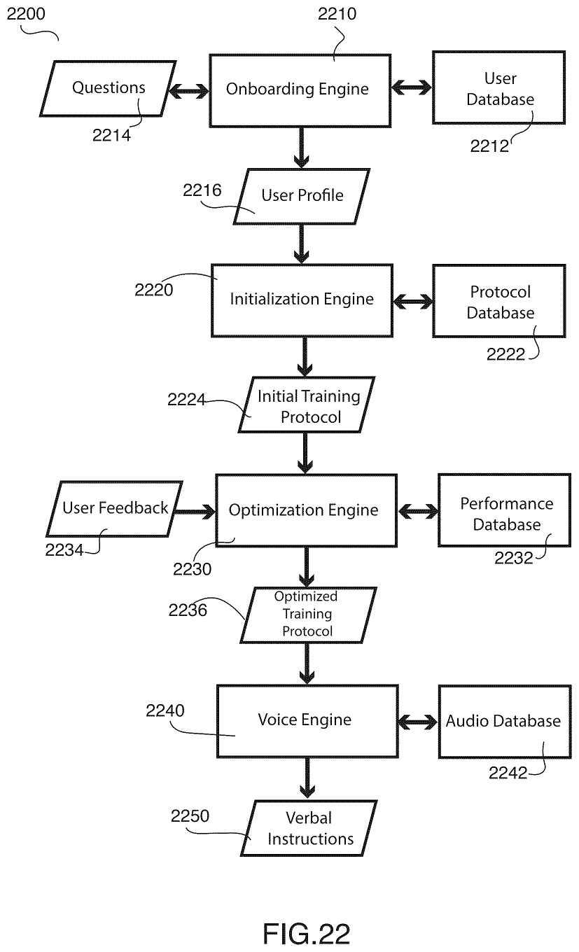

With reference to FIG. 22, depicted is a block diagram of an exemplary system 2200 as used in embodiments of the disclosed invention. The system performs automated onboarding of users by using an onboarding engine 2210 that includes an AI/ML model trained on a database 2212 containing data derived from SME knowledge. The AI/ML model, in this case, a large language model (LLM) is used to perform a dynamic and customized interview to onboard a new user. The LLM is trained using the experiential information gained from human performance improvement coaching. Coaches in the field have amassed data related to client intake, including question substance, question sequence, and question wording, that best identify performance blockages and training programs to correct those blockages. An LLM trained on such a database is used to perform a user intake interview comprised of a series of questions 2214 that is adjusted in response to the user's answers with the objective of identifying the user's performance blockages and training needs without the need for human intervention. The user's answers are collected in a user profile 2216 for the user.

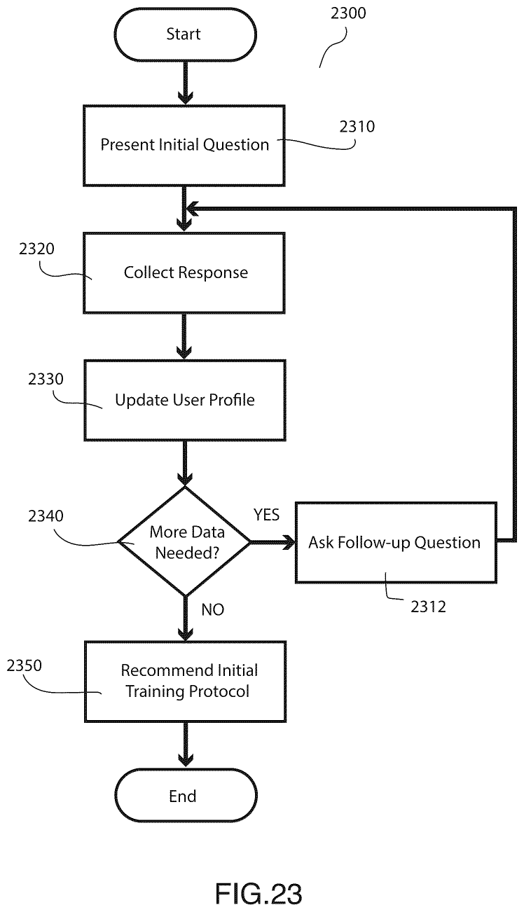

With reference to FIG. 23 is depicted a flow chart showing an exemplary process of performing onboarding 2300 as used in embodiments of the disclosed invention. The onboarding engine has a purpose of eliciting information from the user in an efficient manner so that the user's performance blockage and accompanying training needs can be identified. The engine may use an onboarding question bank that includes a series of questions necessary to collect the required information and sequences through a subset of these questions based on the user's responses. Alternatively, the engine begins with a set of required data and generates appropriate questions using an LLM based on collected user information.

In either case, when commencing the onboarding process, the onboarding engine would start with an initial question 2310. An initial question may be a broad question intended to generally classify the user and set the stage for follow-up questions. For example, the engine may first ask the user “What sport do you play?” by posting the question in the user interface screen of the app. The user responds by inputting their response into the app's user interface, and the onboarding engine collects the response information 2320. The onboarding engine adds the collected information, if any, to the user profile 2330. Then the onboarding engine assesses the user profile and determines if more information is needed to properly classify the user and match them with a training program 2340. Such an assessment includes, for example, confirming a certain percentage of registers in the user profile are filled with information, or performing an AI/ML predictive analysis of the user's responses to determine if a blockage is identified and to what probability. For example, if the user replies “Basketball,” the onboarding engine would assess that only a single data point had been collected, and that more information was needed. The engine would therefore ask a follow-up question 2312 that accounts for the user's response to the first question, for example, “What's your pregame stress level?” The process would continue with collection of data, updating the user profile based on the data, and determining if another question was necessary. Once sufficient data is collected from the user, the system forwards the user profile to the initialization engine, which selects and recommends an initial training protocol 2350. The sequence of questions would proceed in a dynamic way based on the user's responses to create the user profile

### Initialization Engine

With continued reference to FIG. 22, the system also performs automated generation and modification of training protocols through use of an initialization engine 2220 that includes an AI/ML model trained on a database 2222 containing data derived from SME knowledge. The AI/ML model is trained to identify correlations between user profiles, session content, and positive outcomes. The database includes a library of training pathways or static training protocols that have been validated by an experienced coach, as well as associated performance data from practitioners, including archetypes of practitioners. Archetypes are groups of practitioners that have been observed undergoing performance improvement training, that have similar performance needs or responses, and that may be associated with one or more training pathways that have proven effective for that archetype. The initialization engine matches the user's profile to a training protocol, for example, the profile may be used to associate the user with an archetype, and with a training protocol for that archetype. The initialization engine selects an initial training protocol 2224 from among those for the user's archetype. In some embodiments, the initialization engine adjusts the initial training protocol to generate a custom-designed initial training protocol. The custom training protocol is then delivered to the user via the software application.

### Optimization Engine

The system also performs optimization of initial training protocols through use of an optimization engine 2230 that includes an AI/ML model trained on a database 2232 containing data derived from SME knowledge. The AI/ML model is trained to identify correlations between session content, practitioner progress, and positive outcomes. As the user performs the initial training protocol through the software application, the system tracks the user's compliance with the protocol and receives feedback 2234 from the user on subsequent performance. Based on these factors, the user's performance information is collected into the user performance file and provided as input to the optimization engine. The optimization engine includes an AI/ML model trained on data reflecting the usage and subsequent performance metrics of system users, as curated by expert performance coaches. The AI/ML model uses performance data from the user to predict future training needs and to modify the initial training protocol to create an optimized training protocol that anticipates future training needs and provides periodic adjustment of the training protocol as the user progresses through the program.

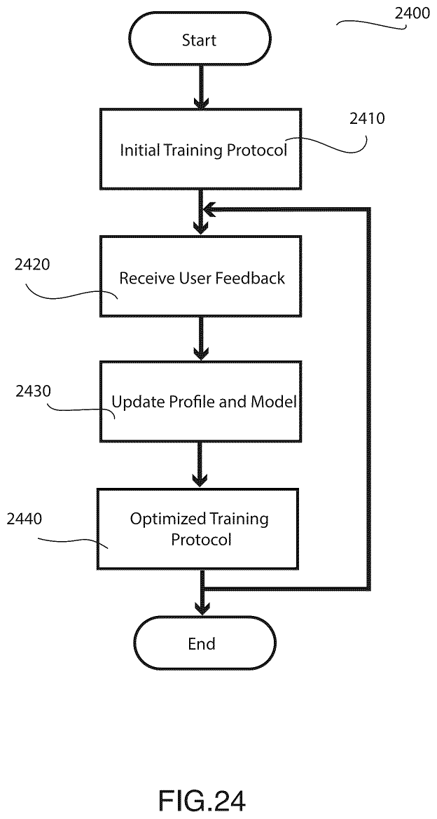

With reference to FIG. 24 is a flow chart depicting an exemplary optimization process 2400 as used in embodiments of the disclosed invention. The user receives an initial training protocol 2410 from the system, participates in the recommended training session, and then performs the time-sensitive activity. For example, a trainee is recommended an initial training protocol for basketball players with performance blockages around shooting free throws. After completing the recommended training protocol, the trainee plays a basketball game and shoots an 80% free throw percentage. The system offers a performance questionnaire through the app's user interface, which the trainee completes. The questionnaire may include the user's assessment of their performance, whether they completed recommended In-Performance training, whether they experienced a blockage, etc. The system uses this feedback to respond to user performance by refining future training protocols to achieve improved results.

The optimization engine receives the feedback provided by the user 2420 and uses the information to update the user's performance profile as well as the AI/ML model 2430. Then the optimization engine uses the updated user profile and AI/ML model to analyze the initial training protocol, and adjust the protocol to improve the user's performance, resulting in an optimized training protocol 2440. For example, the optimization engine may account for the trainee's free throw percentage of 80% and predict that prior training was effective and that major changes are not needed. Alternatively, the engine may observe that the trainee did not perform the recommended In-Performance training and predict that a different In-Performance training sequence would be more effective. The system provides the optimized training protocol to the user through the app. The user then repeats the cycle of participating in the training protocol, performing the time-sensitive activity, and providing feedback to the system through the app. The optimization engine receives the user's feedback 2410 and conducts another optimization cycle. The optimization cycle may be finished once the user experiences a plateau in performance, completes a performance improvement program, chooses to improve a different performance aspect, or other suitable reason.

In some embodiments, the system uses feedback collected by the system to refine future training protocols. For example, the system may record patterns in the user's training, such as time required to perform a tapping sequence. The system may also use access to biometric data, such as heart rate taken by an associated wearable device, to fine tune training protocols for a user.

Through such optimization, the system is able to evolve with the user as training progresses and performance changes. Such content evolution in response to a user's feedback as to their performance in prior systems is only made possible by continuous intervention by a SME. The optimization engine also allows the system to scale up to multiple new users without the need for human intervention.

### Voice Engine

Providing voice instructions to guide a training session by use of a software application is a formidable task. Voice instructions used within the application are recoded from in person training sessions, wherein a coach guides a trainee through a particular session, and the instructions are recorded. Because of the number of training sessions within the system, this process required several months to complete, and the resulting sessions are static and standardized across users. However, use of an AI/ML voice model allows the rapid creation of new training sequences and allows sessions to be customized during a user's performance of a training session.

With continued reference to FIG. 22, the disclosed system is also configured to generate training instructions for a user through use of a voice engine 2240 that includes an AI/ML model, e.g., an LLM, that is trained on an audio database 2242 containing data derived from SME training sessions. The AI/ML model is trained to identify correlations between voice instructions, training protocols, and practitioner behavior. The voice engine uses the model and natural language processing to generate content using synthetic voice rendering based on voice recordings in the audio database.

The audio database contains data including expert statements made during training sessions. The voice LLM, is trained on this audio database, and enables the application to provide voice instructions to a user during a training session. The voice LLM may use a pre-defined static script for a framework, and then dynamically adjust voice instructions based on feedback from the user collected during performance of the training protocol. The system generates new training instructions 2250 customized for a user without the need for human intervention. The voice LLM allows for continuity between sessions, improves quality, and allows the system to be scaled to accommodate new users.

Another benefit of using the disclosed voice model LLM is intuitive cueing or timing of the instructions provided during a training session. For example, during an in-person training session a coach would provide instruction and then pause for the trainee to finish before providing additional instruction. Such timing is difficult to achieve using conventional software applications. However, an individual user's timing patterns may be used to train the AI/ML voice model to provide timely instructions.

Another benefit of the disclosed AI/ML assisted performance improvement system is the use of AI/ML models to integrate other inputs into training protocols. For example, a wearable device may be used to deliver haptic outputs, or earphones may be used to provide sounds to a user during the performance of a training sequence. The timing, intensity, and nature of haptic feedback may be adjusted using AI/ML pattern recognition to improve the effectiveness of the haptic feedback. Likewise, the timing, volume, and type of sound, e.g., the sound of running water, ocean waves, breezes, etc. may be similarly adjusted.

### Specialized Computer System

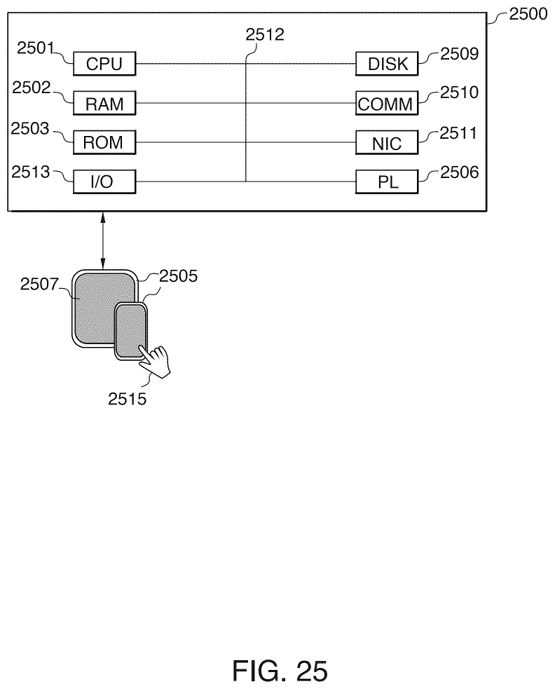

Portions of the disclosed invention may be implemented, at least in part, on a specialized computer system. FIG. 25 is a general block diagram of a special-purpose computer system in which software-implemented processes of the disclosed invention may be embodied. As shown, the system 2500 comprises one or more central processing unit(s) (CPU) or processor(s) 2501 coupled to a random-access memory (RAM) 2502, a read-only memory (ROM) 2503, a fixed storage device 2509 (e.g., hard disk, flash drive), a communication (COMM) port(s) or interface(s) 2510, a high-speed network interface card (NIC) 2511, and a programmable logic card 2506. The system can be accessed by a network-connected computing device, such as a tablet computer or mobile smartphone 2505, having a combined user interface and display device 2507 (e.g., touchscreen), or in some embodiments may be accessed through a software interface, e.g., a web-based software application for providing a performance improvement training program. Although not shown separately, a real time system clock is included with the system, in a conventional manner.

The CPU 2501 comprises a suitable processor for implementing the disclosed invention. The CPU 2501 communicates with other components of the system via a bi-directional system bus or network on a chip module 2512, and any necessary input/output (I/O) controller 2513 circuitry and other “glue” logic. The bus, which includes address lines for addressing system memory, provides data transfer between and among the various components. RAM 2502 serves as the working memory for the CPU 2501. ROM 2504 contains the basic I/O system code (BIOS), which is a set of low-level routines in ROM that application programs and the operating systems can use to interact with the hardware, including reading characters from the keyboard, etc. Some embodiments include a graphics processing unit (GPU) (not shown) to add processing capability to the system.

Mass storage devices 2509 provide persistent storage on fixed and removable media, such as magnetic, optical, or magnetic-optical storage systems, flash memory, or any other available mass storage technology. The mass storage may be shared on a network, e.g., cloud storage, or it may be a dedicated mass storage device. Fixed storage 2509 stores a body of program instructions and data for directing operation of the computer system, including an operating system, user application programs, driver, and other support files, as well as other data files of all sorts. Typically, the fixed storage 2509 serves as the main memory for the system.

In operation, program logic (including that which implements methodology of the disclosed invention described herein) is loaded from fixed storage 2509 into the main (RAM) memory 2502, for execution by the CPU 2501. During operation of the program logic, the system 2500 accepts user input from another computing device, keyboard, mouse, touchscreen, etc. The user interface 2507 permits selection of application programs, entry of software application or keyboard-based input or data, and selection and manipulation of individual data objects represented by a software application or displayed on the display device 2507. Likewise, the pointing device 2515, such as a mouse, or a digit in the case of a touch screen, permits selection and manipulation of objects on the display device. In this manner, these input devices support automatic or manual user input for any process running on the system. In some embodiments, the computer system 2500 displays text and/or graphic images and other data on the display device 2507. A programmable logic module 2506 provides a flexible logic resource for performing certain specialized complex functions.

The system itself communicates with other devices (e.g., other computing devices, or servers) via a port on the NIC 2511 that is connected to a network (e.g., cellular, WIFI, SATCOM, or Ethernet). The system may also communicate with local occasionally connected devices (e.g., serial cable-linked devices) via the COMM interface 2510, which may include a serial port, a Universal Serial Bus (USB) interface, or the like. The mobile computing device operates with wearable devices, such as a fitness tracker, audio device, such as earphones or speakers, haptic devices, such as a smart watch, or other suitable peripheral accessories that communicate with the mobile device through a network, or via a near field communication protocol, such as Bluetooth.

The system may be implemented through various networks and their associated communication devices. Such networks may include servers, modems, or computers, such as a gateway computer or application server which may have access to a cloud computing system. A gateway computer serves as a point of entry into each network and may be coupled to another network by means of a communications link. The gateway may also be directly or indirectly coupled to one or more devices using a communications link, or may be coupled to a storage device such as a data repository or database.

### AI Neural Networks

Embodiments of the disclosed invention use deep neural networks in LLMs and other AI/ML Models described herein to implement the processes described. An exemplary deep neural network includes an input layer, a plurality of hidden layers, and an output layer. In one embodiment, the DNN is a deep auto-encoder neural network (deep ANN) or a convolutional neural network (CNN).

The DNN has two hidden layers, although it is understood that alternative embodiments may have additional hidden layers. Each layer may have one or more nodes (represented by circles in the diagrammatic network). Each node in a current layer is connected to every other node in a previous layer and a next layer. This is referred to as a fully-connected neural network. Other neural network structures are also possible in alternative embodiments of the DNN, in which not every node in each layer is connected to every node in the previous and next layers.

Each node in the input layer can be assigned a value and output that value to every node in the next layer (e.g., hidden layer). The nodes in the input layer can represent features about a particular environment or setting. For example, a DNN used for classifying whether an object is a rectangle may have an input node representing whether the object has flat edges. In this example, assigning a value of 1 to the node may represent that the object does have flat edges and assigning a value of 0 to the node may represent that the object does not have flat edges. In another example, a DNN takes an image as input. In this case, the input nodes may each represent a pixel of the image, such as a pixel of a training image, where the assigned value may represent the intensity of the pixel. Following this example, an assigned value of 1 may indicate that the pixel is completely black and an assigned value of 0 may indicate that the pixel is completely white.

Each node in the hidden layers can receive an outputted value from nodes in a previous layer (e.g., input layer) and associate each of the nodes in the previous layer with a weight. Each hidden node can then multiply each of the received values from the nodes in the previous layer with the weight associated with the nodes in the previous layer and output the sum of the products to each node in the next layer.

Nodes in the output layer handle input values received from the nodes in the hidden layer in a similar fashion. In one example, each output node in the output layer may multiply each input value received from each node in the previous layer (e.g., hidden layer) with a weight and sum the products to generate an output value. The output value of each output node can output information in a predefined format, where the information has some relationship to the corresponding information from the previous layer. Example outputs may include, but are not limited to, classifications, relationships, measurements, instructions, and recommendations. For example, a DNN that classifies whether the object is an ellipse, where an outputted value of 1 from the output node represents that the object is an ellipse and an outputted value of 0 represents that the object is not an ellipse. While the examples provided relate to classifying geometric shapes, this is only for illustrative purposes. The output nodes can also be used to classify any of a wide variety of objects and other features and otherwise output any of a wide variety of desired information in desired formats.

### Training Neural Networks

Once a given network has been structured for a task, the neural network is trained using a training dataset. To begin training the DNN, initial weights may be chosen randomly or by pre-training using a deep belief network. The training cycle can then be performed in either a supervised or unsupervised manner.

Supervised learning uses a training set to teach models to yield the desired output. The training dataset includes inputs and desired outputs, which allow the model to learn over time, or when the training dataset includes input having known output and the output of the neural network is manually graded. The network processes the inputs and compares the resulting outputs against a set of expected or desired outputs. Errors are then propagated back through the system. The training framework can adjust to change the weights that control the untrained neural network. The training framework can provide tools to monitor how well the untrained neural network is converging towards a model suitable for generating correct answers based on known input data. The training process repeatedly occurs as the network weights are adjusted to refine the output generated by the neural network. The training process can continue until the neural network reaches a statistically desired accuracy associated with a trained neural network. The trained neural network can then be deployed to implement any number of machine learning operations to output a result.

Supervised learning is typically separated into two types of problems: classification and regression. Classification uses an algorithm to assign test data accurately into specific categories. Regression is used to understand the relationship between dependent and independent variables. Numerous different algorithms and computation techniques can be used in supervised machine learning, including but not limited to, neural networks, naïve bayes, linear regression, logistic regression, support vector machines (SVM), k-nearest neighbor, and random forest.

Unsupervised learning is a learning method in which the network uses algorithms to analyze and cluster unlabeled data. These algorithms discover hidden patterns or data groupings. Therefore, the training dataset includes input data without any associated output data. The untrained neural network can learn groupings within the unlabeled input and determine how individual inputs relate to the overall dataset. Unsupervised training can be used to for three main tasks: clustering, association, and dimensionality. Clustering is a data mining technique that groups unlabeled data based on similarities and differences. This technique is often used to process raw, unclassified data objects into groups represented by structures or patterns in the information. Association is a rule-based method for finding relationships between variables in a given dataset. This method is often used for market basket analysis. Dimensionality reduction is used when a given dataset's number of features (dimensions) is too high. This technique is commonly used in the preprocessing of data.

Variations of supervised and unsupervised training may also be employed. Semi-supervised learning is a technique in which the training dataset includes a mix of labeled and unlabeled data of the same distribution. Incremental learning is a variant of supervised learning in which input data is continuously used to train the model further. Incremental learning enables the trained neural network to adapt to the new data without forgetting the knowledge instilled within the network during initial training.

### Natural Language Processing

Embodiments of the disclosed invention use a natural language processing platform for automated training and performance evaluation, such as that performed by the voice engine described herein. A natural language processing (NLP) platform can be configured to perform NLP processing techniques in audio transcript file analysis to identify one or more events (e.g., ask for the sale, multiline offer, objection handling, or selling effort time) and/or evaluate performance based on the audio transcript files. Additionally, the NLP platform can maintain a model for dynamic performance evaluation and training that the NLP platform may use to analyze the audio transcript files. The NLP platform can dynamically update the audio transcript files as additional audio transcript files and/or performance data are received. In another example, the NLP platform can be associated with an organization or entity (e.g., an insurance agency, or the like).

The NLP platform may have instructions that direct and/or cause the NLP platform to execute advanced natural language claim processing techniques. For example, the NLP platform can apply NLP techniques to identify features in one or more audio transcription files indicative of one or more performance events and/or additional metrics associated with the audio transcription files.

Memory used by the NLP platform may include several components or modules. An NLP database may store information used by the NLP module and/or the NLP platform in transcription analysis, evaluating performance, providing performance feedback, and/or in performing other functions. Machine learning engine may have instructions that direct and/or cause the NLP platform to perform transcription analysis, evaluate performance, provide performance feedback, and to set, define, and/or iteratively refine optimization rules and/or other parameters used by the NLP platform and/or other systems.

### Automatic Speech Recognition

Embodiments of the disclosed invention use an automatic speech recognition processing of frequent phrases or words. In one embodiment, a computing device receives an audio input from a user. The computing device compares the audio input to one or more speech recognition models for frequent phrases or words and determines whether the audio input includes a frequent phrase or word. When the computing device determines that the audio input includes a frequent phrase, the computing device processes the frequent phrase by executing a command corresponding with the phrase. When the computing device determines that the audio input does not include a frequent phrase, the computing device forwards the audio input to an ASR module After the ASR module performs speech recognition on the audio input, the speech recognition results can be used for further operations.

The ASR module can also process spoken audio data into text by transcribing audio data into text data representing the words of the speech contained in the audio data. The text data can then be used for various purposes, such as executing system commands, inputting data, etc. Audio data, including spoken utterances, can be processed in real time or saved and processed later. A spoken utterance in the audio data is input to the ASR module, which then interprets the utterance based on the similarity between the utterance and models known to the ASR module. For example, the ASR module may compare the input audio data with models for sounds (e.g., speech units or phonemes) and sequences of sounds to identify words and phrases that match the sequence of sounds spoken in the utterance of the audio data. The different ways a spoken utterance may be interpreted may each be assigned a probability or a recognition score representing the likelihood that a particular set of words matches those spoken in the utterance. The recognition score may be based on several factors including, for example, the similarity of the sound in the utterance to models for language sounds (e.g., an acoustic model) and the likelihood that a particular word that matches the sounds would be included in the sentence at the specific location (e.g., using a language or grammar model). Based on these factors, the ASR module may output the most likely words or phrases recognized in the audio data.

The components for processing spoken audio data into text of the ASR module can include an acoustic front end (AFE), a speech recognition engine, and speech storage. The AFE can receive audio data and transform it into data for processing by the speech recognition engine. The speech recognition engine compares the speech recognition data with the acoustic, language, and other data models and information stored in the speech storage for recognizing the speech contained in the original audio data. The AFE may also reduce noise in the audio data, identify parts of the audio data containing speech for processing, and segment and process the identified speech components.

The speech recognition engine can process the output from the AFE about information stored in the speech storage. The speech storage can include a variety of information for speech recognition, such as data matching pronunciations of phonemes to particular words and/or phrases. This data may be referred to as an acoustic model. The speech storage can also include a dictionary of words or a lexicon and can also include data describing words and/or phrases that are likely to be used together in particular contexts. This data may be referred to as a language or grammar model. Other information may also be stored in the speech storage for use in speech recognition by the speech recognition engine in conjunction with language phonemes, words, and/or phrases as known in the speech storage. The contents of the speech storage can be prepared for general ASR use or may be customized to include sounds, words, and phrases that are likely to be used in a particular application, such as common instructions provided by performance improvement coaches. The speech recognition engine can use many different techniques to match information to phonemes. For example, a Hidden Markov Models (HMMs) may be used as a technique.

This has been a description of the disclosed invention along with a preferred method of practicing the invention, however the scope of the inventions ought to be determined by the appended claims.

What is claimed is:

1. A system, comprising:
a software application comprising:
an onboarding engine for collecting information from the user to create a user profile for a user,
an initialization engine for generating an initial training protocol for the user,
an optimization engine for modifying the initial training protocol to create an optimized training protocol, and
a voice engine for providing voice instructions to guide the user through the initial training protocol and the optimized training protocol;
a computer for running the software application, the computer comprising a processor, processor associated memory, a user interface, and data storage, wherein the software application exchanges information with the user via interaction with the interface, the application controlling the interface by presenting outputs on a display and reading data from the interface in relation to the outputs; and
a user file, wherein the user file stores information about the user, including the user profile, and wherein the user file is accessible by the application.

2. The system of claim 1, wherein each of the onboarding engine, the initialization engine, the optimization engine, and the voice engine further comprises an artificial intelligence or machine learning model trained on a database that includes data validated by a subject matter expert (SME).

3. The system of claim 1, wherein the onboarding engine dynamically selects questions for the user, wherein the questions elicit information to populate a user profile, and wherein the information is used to identify a performance blockage for the user.

4. The system of claim 3, wherein the initialization engine matches the user profile with an initial training protocol, wherein the initial training protocol addresses the performance blockage.

5. The system of claim 1, wherein the initialization engine associates the user profile with an archetype, and wherein the archetype is associated with one or more training pathways.

6. The system of claim 1, wherein the optimization engine modifies the initial training protocol based on user feedback, wherein the feedback includes performance of a time-sensitive activity.

7. The system of claim 6, wherein the feedback includes biometric data collected by a wearable device.

8. The system of claim 1, wherein the voice engine generates synthetic voice instructions using a natural language processor.

9. The system of claim 1, wherein the voice engine allows the creation of new training instructions that are customized to the user.

10. The system of claim 1, further comprising a haptic device for providing haptic outputs to the user, and an audio device for providing audio outputs to the user, wherein the haptic outputs and audio outputs are integrated with the initial training protocol and the optimized training protocol.

11. A method, comprising:
onboarding a user using an onboarding engine that dynamically generates questions to elicit information for a user profile;
selecting an initial training protocol using an initialization engine that matches the user profile to the initial training protocol;
modifying the initial training protocol to create an optimized training protocol using an optimization engine that accesses feedback from the user; and
guiding the user to perform the initial training protocol and the optimized training protocol with voice instructions generated by a voice engine.

12. The method of claim 11, wherein each of the onboarding engine, the initialization engine, the optimization engine, and the voice engine further comprises an artificial intelligence or machine learning model trained on a database that includes data validated by an SME.

13. The method of claim 11, wherein the onboarding step further comprises identifying a performance blockage for the user.

14. The method of claim 13, wherein the initial training protocol addresses the performance blockage.

15. The method of claim 11, wherein the selecting step further comprises associating the user profile with an archetype, wherein the archetype is associated with one or more training pathways.

16. The method of claim 11, wherein the feedback includes information about the user's performance of a time-sensitive activity.

17. The method of claim 11, wherein the feedback includes one of timing data, or biometric data.

18. The method of claim 11, wherein the optimization step further comprises periodically modifying the optimized training protocol based on feedback from the user.

19. The method of claim 11, wherein the guiding step further comprises generating synthetic voice instructions based on an audio database that contains recordings of voice instructions issued in a training session.

20. The method of claim 11, wherein the voice engine generates new training instructions to implement the optimized training protocol.
